(19) United States
(12) Patent Application Publication
(10) Pub. No.: US 2026/0315416 A1
(43) Pub. Date: Oct. 15, 2026

(54) PHENYLLACTIC ACID COMPOSITIONS AND RELATED METHODS OF USE

(71) Applicant: Arizona Board of Regents on Behalf of the University of Arizona, Tucson, AZ (US)

(72) Inventors: Melissa HERBST-KRALOVETZ, Tucson, AZ (US); Abhijit DATE, Tucson, AZ (US); Pawel LANIEWSKI, Tucson, AZ (US); Yogesh SUTAR, Tucson, AZ (US)

(21) Appl. No.: 19/476,406

(22) PCT Filed: Apr. 22, 2024
(86) PCT No.: PCT/US2024/025731

§ 371 Date: Oct. 17, 2025

Related U.S. Application Data
(60) Provisional application No. 63/497,724, filed on Apr. 22, 2023.

(51) Int. Cl. A61K 31/192; A61K 47/18; A61P 15/02; A61P 31/04

(52) U.S. Cl. CPC A61K 31/192; A61K 47/186; A61P 15/02; A61P 31/04

(57) ABSTRACT

This invention is in the field of medicinal chemistry. In particular, the invention relates to compositions comprising phenyllactic acid, and related methods for treating, preventing, and/or ameliorating symptoms and/or conditions characterized with microbiome dysbiosis (e.g., sexually transmitted infections, urinary tract infections, bacterial vaginosis).

The present application claims priority to U.S. Provisional Application No. 63/497,724, filed Apr. 22, 2023, which is incorporated herein by reference in its entirety.

### FIELD OF THE INVENTION

This invention is in the field of medicinal chemistry. In particular, the invention relates to compositions comprising phenyllactic acid, and related methods for treating, preventing, and/or ameliorating symptoms and/or conditions characterized with microbiome dysbiosis (e.g., sexually transmitted infections, urinary tract infections, bacterial vaginosis).

### INTRODUCTION

Phenyllactic acid (3-phenyllactic acid or 2-hydroxy-3-phenylpropanoic acid;

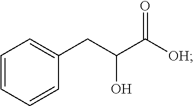 [SMILES: O=C(O)C(O)Cc1ccccc1]

PLA) is an organic phenolic acid, naturally found in honey and foods and produced by lactic acid bacteria (LAB).¹ It is a common metabolite of many LABs, including Lactobacillus species, which are widely found in the healthy microbiomes of the human gut², bladder³, and vagina.⁴ This metabolite gained significant interest as a “green preservative” in the food industry^{5, 6} owing to its natural broad-spectrum antimicrobial activity against bacteria⁷⁻¹⁰ and fungi.^{11, 12} PLA exists in two optical isomeric forms known as enantiomers, D-PLA and L-PLA, with D-PLA demonstrating greater antimicrobial activity than L-PLA.¹³ Beyond the food industry, PLA has been researched for vast applications in agriculture¹⁴, pharmaceutical¹⁵, and cosmetic¹⁶ industries. In addition, PLA has been investigated in the biomedical field, where it has demonstrated beneficial metabolic functions, including immunomodulatory¹⁷ and anti-inflammatory activity.¹⁸

Recently, it was discovered that commensal vaginal Lactobacillus species, including Lactobacillus crispatus, Lactobacillus paragasseri, and Lactobacillus iners, were capable of producing PLA in vitro.^{19,20} Within the lower female reproductive tract, it is well established that microbiota dominated by Lactobacillus spp. provide protection against pathogenic diseases, such as sexually transmitted infections^{21,22}, urinary tract infections²³, and bacterial vaginosis.^{24, 25} Lactobacilli achieve this by producing lactic acid to maintain an acidic microenvironment.²⁶ However, the role of other metabolites produced by lactobacilli, such as PLA and other aromatic lactic acids, is still not well-known. An in vitro study showed that PLA is produced by a bladder isolate of L. crispatus and exhibits antimicrobial properties against gram-positive, gram-negative, and fungal urogenital pathogens, such as Streptococcus agalactiae (also known as Group B Streptococcus), Klebsiella pneumoniae, and Candida albicans.²⁷ PLA's broad-spectrum antimicrobial properties, potential immunomodulatory functions, and its natural production in the cervicovaginal microenvironment demonstrate its promising applications as an intravaginally delivered compound to maintain mucosal homeostasis in the lower female reproductive tract. However, PLA has low kinetic solubility in water, and it requires toxic solvents such as dimethyl sulfoxide (DMSO) for the solubilization of high quantities of PLA. Indeed, DMSO cannot be used for vaginal delivery.

As such, a solubilization strategy that uses biocompatible ingredients to solubilize high quantities of PLA for intravaginal delivery is required.

The present invention addresses these needs.

### SUMMARY

Ionic liquids (ILs) are organic salts, composed solely of cations and anions, with melting points lower than 100° C. In the past few years, ILs have received great attention in the pharmaceutical and biomedical sciences as a novel delivery strategy to improve the solubility, permeability, and bioavailability of ionizable compounds.^{28, 29} We hypothesised that anionic PLA can interact with biocompatible cations to yield biocompatible and highly soluble PLA ILs suitable for delivery to various mucosal surfaces including the vagina.

Experiments conducted during the course of developing embodiments for the present invention demonstrated that crystalline PLA can be transformed into amorphous ionic liquids (PLA ILs), using cations of natural origin (choline and carnitine), that have ~55-fold higher water solubility compared to D- and L-PLA. In addition, in vitro cytotoxicity testing of these PLA ILs on human cervical epithelial cells determined their safety.

Accordingly, the present invention relates to compositions comprising phenyllactic acid, and related methods for treating, preventing, and/or ameliorating symptoms and/or conditions characterized with microbiome dysbiosis (e.g., sexually transmitted infections, urinary tract infections, bacterial vaginosis).

In certain embodiments, the present invention provides compositions comprising phenyllactic acid

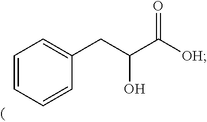 [SMILES: O=C(O)C(O)Cc1ccccc1]

PLA); including pharmaceutically acceptable salts, hydrates, solvates, stereoisomers, and/or deuterated analogues thereof. In some embodiments, the PLA is either the D enantiomer form of PLA, the L enantiomer form of PLA, or a mixture thereof. In some embodiments, the composition is configured for topical or mucosal delivery to a subject. In some embodiments, the subject is a human subject (e.g., a female human subject). In some embodiments, the composition is a liquid, wherein the PLA is solubilized within the liquid. In some embodiments, the composition is an ionic liquid, wherein the PLA is solubilized within the ionic liquid.

In some embodiments, the PLA is associated with choline, wherein the PLA is associated

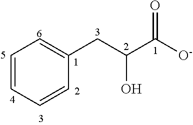 [SMILES: O=C([O-])C(O)Cc1ccccc1]

with choline in the following manner:

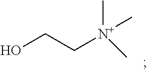

and/or the PLA is associated with carnitine, wherein the PLA is associated with carnitine in the following manner:

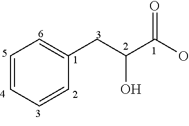 [SMILES: O=C(O)C(O)Cc1ccccc1]

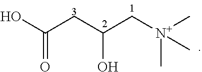

In some embodiments, the ratio of PLA to choline is approximately between 1:1 to 2:1. In some embodiments, the ratio of PLA to carnitine is approximately between 1:1. In some embodiments, the composition is a liquid or ionic liquid, wherein the PLA associated with choline and/or carnitine is solubilized in the liquid or ionic liquid.

In some embodiments, the composition is a pharmaceutical composition.

In certain embodiments, the present invention provides methods for increasing the amount of PLA within a subject's microbiome through administering to a subject a therapeutically effective amount of a composition comprising PLA as described herein. In some embodiments, the subject is a human subject.

In certain embodiments, the present invention provides methods for elevating the richness and diversity of a subject's microbiome through administering to a subject a therapeutically effective amount of a composition comprising PLA as described herein.

In certain embodiments, the present invention provides methods for treating or preventing microbiome dysbiosis in a subject through administering to a subject a therapeutically effective amount of a composition comprising PLA as described herein.

In certain embodiments, the present invention provides methods for treating or preventing a condition associated with diminished or inhibited commensal vaginal Lactobacillus species activity and/or expression in a subject through administering to a subject a therapeutically effective amount of a composition comprising PLA as described herein.

In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the subject is suffering from or at risk of suffering from a condition (e.g., sexually transmitted infections (e.g., syphilis, gonorrhoea, chlamydia, trichomoniasis, hepatitis B, herpes simplex virus (HSV), HIV, and human papillomavirus (HPV)), urinary tract infections, and bacterial vaginosis) associated with inhibited or diminished PLA expression and/or activity and/or levels within the subject's microbiome. In some embodiments, the microbiome is a cervico-vaginal microbiome. In some embodiments, the subject is experiencing diminished or inhibited commensal vaginal Lactobacillus species activity and/or expression. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression.

In certain embodiments, the present invention provides methods for treating or preventing a sexually transmitted infection associated with diminished or inhibited commensal vaginal Lactobacillus species activity and/or expression in a subject through administering to a subject a therapeutically effective amount of a composition comprising PLA as described herein. In some embodiments, the subject is a human subject. In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the diminished or inhibited commensal vaginal Lactobacillus species activity and/or expression is in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression.

In certain embodiments, the present invention provides methods for treating or preventing a urinary tract infection associated with diminished or inhibited commensal vaginal Lactobacillus species activity and/or expression in a subject through administering to a subject a therapeutically effective amount of a composition comprising PLA as described herein. In some embodiments, the subject is a human subject. In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the diminished or inhibited commensal vaginal Lactobacillus species activity and/or expression is in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression.

In certain embodiments, the present invention provides methods for treating or preventing bacterial vaginosis associated with diminished or inhibited commensal vaginal Lactobacillus species activity and/or expression in a subject through administering to a subject a therapeutically effective amount of a composition comprising PLA as described herein. In some embodiments, the subject is a human subject. In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the diminished or inhibited commensal vaginal Lactobacillus species activity and/or expression is in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression.

In certain embodiments, the present invention provides methods for treating or preventing cervico-vaginal dysbiosis in a subject through administering to a subject a therapeutically effective amount of a composition comprising PLA as described herein. In some embodiments, the subject is a human subject. In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the subject is experiencing diminished or inhibited commensal vaginal Lactobacillus species activity and/or expression in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression. In some embodiments, the cerivo-vaginal dysbiosis is related to one or more of Gardnerella vaginalis, Prevotella bivia, and Atopobium vaginae.

In certain embodiments, the present invention provides a method for treating, preventing, and/or reducing dysbiotic vaginal bacteria in a subject through administering to a subject a therapeutically effective amount of a composition comprising PLA as described herein, wherein the administering is to the cervicovaginal mucosa of the subject, wherein the administering is followed by donor engraftment of the cervicovaginal mucosa, vaginal microbiota transplant of the cervicovaginal mucosa, and/or probiotic colonization of the cervicovaginal mucosa. In some embodiments, the subject is a human subject. In some embodiments, the subject is a female human subject. In some embodiments, the subject is experiencing diminished or inhibited commensal vaginal Lactobacillus species activity and/or expression in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression. In some embodiments, the cerivo-vaginal dysbiosis is related to one or more of Gardnerella vaginalis, Prevotella bivia, and Atopobium vaginae.

In certain embodiments, the present invention provides methods for treating, preventing, and/or reducing dysbiotic vaginal bacteria in a subject through administering to a subject one or more of a donor engraftment of the cervicovaginal mucosa, vaginal microbiota transplant of the cervicovaginal mucosa, and a probiotic colonization of the cervicovaginal mucosa, wherein the donor engraftment, vaginal microbiota transplant, and the probiotic colonization each comprise a therapeutically effective amount of a composition comprising PLA as described herein, wherein the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the subject is experiencing diminished or inhibited commensal vaginal Lactobacillus species activity and/or expression in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression. In some embodiments, the cerivo-vaginal dysbiosis is related to one or more of Gardnerella vaginalis, Prevotella bivia, and Atopobium vaginae.

### BRIEF DESCRIPTION OF THE DRAWINGS

FIG. 1 provides synthesis scheme for the development of (A) Chol:D-PLA (1:1) and Chol:L-PLA (1:1), (B) Chol:D-PLA (1:2) and Chol:L-PLA (1:2), and (C) Car:D-PLA (1:1) and Car:L-PLA (1:1).

FIG. 2: FTIR characterization of D- and L-PLA, carnitine and its corresponding ILs. The FTIR spectra of PLA ILs show disappearance of or significant shifts in —C═O stretch of PLA indicating its interaction with the choline and carnitine base.

FIG. 3: ¹H NMR spectra of D-PLA ILs (A) and L-PLA ILs (B) showed disappearance of carboxylic acid proton shift of PLA (—COOH: 1). Whereas significant displacement of hydroxyl proton (—OH: 3) and proton adjacent to hydroxyl group (—CH: 2) of PLA was observed in all ¹H NMR spectrum of PLA ILs. This indicates the ionic interaction between D and L-PLA and counterions.

FIG. 4: The overlay HPLC chromatogram of D-PLA, L-PLA, Chol:D-PLA (1:1), Chol:L-PLA (1:1), Chol:D-PLA (1:2), Chol:L-PLA (1:2), Car:D-PLA and Car:L-PLA. (concentration: 60 μM).

FIG. 5: (A) Thermogravimetric analysis (TGA) of PLA, carnitine and its corresponding ILs. Chol:D-PLA (1:2) and Chol:L-PLA (1:2) displayed higher thermal stability compared to pure PLA. (B) T5% (onset temperature at 5% mass loss) of PLA and its ILs observed in the TGA analysis. (C) Differential thermal colorimetry (DSC) thermogram of D- and L-PLA and its ILs. All the samples were set up to their T5%. Disappearance of PLA melting peak in its ILs indicates the amorphization of PLA because of interaction between PLA and counterions. (D) Glass transition temperature (Tg) analysis of D- and L-PLA and its choline/carnitine based ILs.

FIG. 6: X-ray diffractogram of D- and L-PLA, carnitine free base, and its corresponding ILs. All the PLA ILs showed the absence of characteristic X-ray diffraction peaks corresponding to PLA and carnitine which confirms the formation of IL with full amorphization.

FIG. 7: Table 1: Solubility of D/L-PLA and their ILs in water. Data expressed as mean±standard deviation (S.D.); n=3.

FIG. 8: PLA ILs causes no morphological changes to human cervical epithelial cells at low concentrations (≤12.5 mM). Microscopy images, at ×10 magnification, of crystal violet stained cervical epithelial (A2EN) cells, grown as monolayers, following 24-hr treatment with varying concentrations of (A) Chol:D-PLA (1:1) and Chol:L-PLA (1:1), (B) Chol:D-PLA (1:2) and Chol:L-PLA (1:2), and (C) Car:D-PLA (1:1) and Car:L-PLA (1:1), and (D) HCl control solutions. Deionised water and 1% Conceptrol were used as negative (-ve) and positive (+ve) controls, respectively.

FIG. 9: PLA ILs causes no cytotoxicity to human cervical epithelial cells at low concentrations (≤6.25 mM). Percentage cell viability was determined by MTT assay following 24-hr treatment of cervical epithelial (A2EN) cell monolayers with varying concentrations of PLA-ILs and pH solutions: (A) Chol:D-PLA (1:1) and Chol:L-PLA (1:1), (B) Chol:D-PLA (1:2) and Chol:L-PLA (1:2), and (C) Car:D-PLA (1:1) and Car:L-PLA (1:1). Absorbance was measured at 570 nm (650 nm reference). Percentage cell viability was calculated relative to untreated control (100%) and plotted as mean±SEM, using at least 3 independent experiments per compound. Significance was determined using two-way ANOVA with Bonferroni's adjustment. Asterisks indicate significance: ****P<0.0001; ***P<0.001; **P<0.01; *P<0.05.

FIG. 10: Low pH of HCl control solutions are cytotoxic to human epithelial cells. Percentage cell viability was determined by MTT assay following 24-hr treatment of cervical epithelial (A2EN) cell monolayers with HCl control solutions ranging from 4-7.5 pH. Absorbance was measured at 570 nm (650 nm reference). Percentage cell viability was calculated relative to untreated control (100%) and plotted as mean±SEM, using 6 independent experiments per pH solution.

FIGS. 11-22 provide ¹H NMR spectrum for various associations of PLA and either choline or carnitine.

FIG. 23: PLA-ILs inhibit growth of vaginal bacteria differently based on the agar medium, the IL cation, and the PLA enantiomer. The zone of inhibition of L. crispatus and P. bivia on both their optimum medium and NYC III medium, was measured following treatment with disks immersed in IM PLA-IL. 10% bleach was used as a positive control. Plates were incubated for 24 hrs at 37° C. in anaerobic conditions.

FIG. 24: The growth of health- and BV-associated bacteria are inhibited more strongly by compounds #1 and #2 PLA (1:2) than by compound #2 PLA (1:1). (A) The MIC for each PLA-IL to inhibit the growth of vaginal bacteria in NYC III broth was measured. Each PLA-IL was serially diluted in the broth and inoculated with bacterial suspension adjusted to OD₆₀₀ of 0.05 (grey dashed line). The OD₆₀₀ was then measured 22 hours post bacterial inoculation. Black arrows indicate MIC. Data plotted as mean+SEM, with 2-3 replicates per compound. (B) Bar graph of the MICs for each compound.

FIG. 25: Compound #1 PLA does not cause any morphological changes to human cervical epithelial cells at concentrations ≤12.5 mM, and compound #2 PLA (1:2) at 25 mM. Microscopy images, at ×10 magnification, of crystal violet stained cervical epithelial cells, grown as monolayers, following treatment with each PLA-IL. pH values of the cell supernatant shown under the images. 1% conceptrol and H₂O were used as positive and negative controls, respectively. At high PLA-IL concentrations, either the PLA-IL or low pH may have caused cell stress and death.

FIG. 26: Compound #1 PLA does not cause any cytotoxicity to human cervical epithelial cells at concentrations ≤6.25 mM, and compound #2 PLA (1:2) at 12.5 mM. Cervical epithelial cells were treated with increasing concentrations of each PLA-IL, incubated for 24 hrs, and then the MTT assay was conducted. Absorbance was measured at 570 nm and subtracted from 650 nm reference. Percentage cell viability was calculated and plotted as mean+SEM, with 9 replicates per condition. Significance was determined using two-way ANOVA with Bonferroni's adjustments. Asterisks indicate significance (****P<0.0001; ***P<0.001; **P<0.0; *P<0.05).

### DETAILED DESCRIPTION OF THE INVENTION

Phenyllactic acid (PLA), is a naturally produced, broad-spectrum antimicrobial compound with activity against bacteria and fungi. PLA can be produced by a variety of lactic acid bacteria, including vaginal Lactobacillus species, which are healthy constituents of the vaginal microbiome with a protective role against invading pathogenic bacteria and/or fungi. Additionally, PLA has been shown to exhibit anti-inflammatory and immunomodulatory properties, overall indicating its therapeutic potential as an intravaginally delivered compound for the vaginal microbiome modulation. However, PLA has low kinetic solubility in water, dissolving only in toxic solvents. Hence, strategies to improve the solubility of PLA are necessary to facilitate its intravaginal delivery. Using biocompatible cations, choline, and carnitine, experiments described herein transformed both D- and L-enantiomers of crystalline PLA into amorphous low-melting ionic liquids (ILs) with high water solubility. Such experiments further evaluated the in vitro cytotoxicity of PLA ILs to human cervical epithelial cells. Microscopic visualisation of cellular morphology using crystal violet staining and MTT cell proliferation assay revealed that PLA ILs result in minimal morphological changes and low cytotoxicity to human cervical epithelial cells. Overall, such experiments demonstrated that transforming PLA into ILs efficiently enhances its solubility in water and these formulations are not toxic to human epithelial cells.

Experiments conducted during the course of developing embodiments for the present invention demonstrated that crystalline PLA can be transformed into amorphous ionic liquids (PLA ILs), using cations of natural origin (choline and carnitine), that have ~55-fold higher water solubility compared to D- and L-PLA. In addition, in vitro cytotoxicity testing of these PLA ILs on human cervical epithelial cells determined their safety.

Accordingly, the present invention relates to compositions comprising phenyllactic acid, and related methods for treating, preventing, and/or ameliorating symptoms and/or conditions characterized with microbiome dysbiosis (e.g., sexually transmitted infections, urinary tract infections, bacterial vaginosis).

In certain embodiments, the present invention provides compositions comprising phenyllactic acid

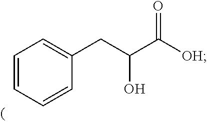 [SMILES: O=C(O)C(O)Cc1ccccc1]

PLA); including pharmaceutically acceptable salts, hydrates, solvates, stereoisomers, and/or deuterated analogues thereof. In some embodiments, the PLA is either the D enantiomer form of PLA, the L enantiomer form of PLA, or a mixture thereof. In some embodiments, the composition is configured for topical or mucosal delivery to a subject. In some embodiments, the subject is a human subject (e.g., a female human subject). In some embodiments, the composition is a liquid, wherein the PLA is solubilized within the liquid. In some embodiments, the composition is an ionic liquid, wherein the PLA is solubilized within the ionic liquid.

In some embodiments, the PLA is associated with choline, wherein the PLA is associated

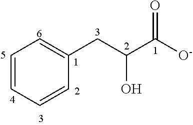 [SMILES: O=C([O-])C(O)Cc1ccccc1]

with choline in the following manner:

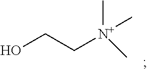

and/or the PLA is associated with carnitine, wherein the PLA is associated with carnitine in the following manner:

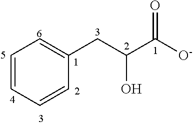 [SMILES: O=C([O-])C(O)Cc1ccccc1]

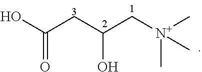

In some embodiments, the ratio of PLA to choline is approximately between 1:1 to 2:1. In some embodiments, the ratio of PLA to carnitine is approximately between 1:1. In some embodiments, the composition is a liquid or ionic liquid, wherein the PLA associated with choline and/or carnitine is solubilized in the liquid or ionic liquid.

In some embodiments, the composition is a pharmaceutical composition.

In certain embodiments, the present invention provides methods for increasing the amount of PLA within a subject's microbiome through administering to a subject a therapeutically effective amount of a composition comprising PLA as described herein. In some embodiments, the subject is a human subject.

In certain embodiments, the present invention provides methods for elevating the richness and diversity of a subject's microbiome through administering to a subject a therapeutically effective amount of a composition comprising PLA as described herein.

In certain embodiments, the present invention provides methods for treating or preventing microbiome dysbiosis in a subject through administering to a subject a therapeutically effective amount of a composition comprising PLA as described herein.

In certain embodiments, the present invention provides methods for treating or preventing a condition associated with diminished or inhibited commensal vaginal Lactobacillus species activity and/or expression in a subject through administering to a subject a therapeutically effective amount of a composition comprising PLA as described herein.

In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the subject is suffering from or at risk of suffering from a condition (e.g., sexually transmitted infections (e.g., syphilis, gonorrhoea, chlamydia, trichomoniasis, hepatitis B, herpes simplex virus (HSV), HIV, and human papillomavirus (HPV)), urinary tract infections, and bacterial vaginosis) associated with inhibited or diminished PLA expression and/or activity and/or levels within the subject's microbiome. In some embodiments, the microbiome is a cervico-vaginal microbiome. In some embodiments, the subject is experiencing diminished or inhibited commensal vaginal Lactobacillus species activity and/or expression. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression.

In certain embodiments, the present invention provides methods for treating or preventing a sexually transmitted infection associated with diminished or inhibited commensal vaginal Lactobacillus species activity and/or expression in a subject through administering to a subject a therapeutically effective amount of a composition comprising PLA as described herein. In some embodiments, the subject is a human subject. In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the diminished or inhibited commensal vaginal Lactobacillus species activity and/or expression is in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression.

In certain embodiments, the present invention provides methods for treating or preventing a urinary tract infection associated with diminished or inhibited commensal vaginal Lactobacillus species activity and/or expression in a subject through administering to a subject a therapeutically effective amount of a composition comprising PLA as described herein. In some embodiments, the subject is a human subject. In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the diminished or inhibited commensal vaginal Lactobacillus species activity and/or expression is in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression.

In certain embodiments, the present invention provides methods for treating or preventing bacterial vaginosis associated with diminished or inhibited commensal vaginal Lactobacillus species activity and/or expression in a subject through administering to a subject a therapeutically effective amount of a composition comprising PLA as described herein. In some embodiments, the subject is a human subject. In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the diminished or inhibited commensal vaginal Lactobacillus species activity and/or expression is in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression.

In certain embodiments, the present invention provides methods for treating or preventing cervico-vaginal dysbiosis in a subject through administering to a subject a therapeutically effective amount of a composition comprising PLA as described herein. In some embodiments, the subject is a human subject. In some embodiments, the subject is a female human subject. In some embodiments, the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the subject is experiencing diminished or inhibited commensal vaginal Lactobacillus species activity and/or expression in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression. In some embodiments, the cerivo-vaginal dysbiosis is related to one or more of Gardnerella vaginalis, Prevotella bivia, and Atopobium vaginae.

In certain embodiments, the present invention provides a method for treating, preventing, and/or reducing dysbiotic vaginal bacteria in a subject through administering to a subject a therapeutically effective amount of a composition comprising PLA as described herein, wherein the administering is to the cervicovaginal mucosa of the subject, wherein the administering is followed by donor engraftment of the cervicovaginal mucosa, vaginal microbiota transplant of the cervicovaginal mucosa, and/or probiotic colonization of the cervicovaginal mucosa. In some embodiments, the subject is a human subject. In some embodiments, the subject is a female human subject. In some embodiments, the subject is experiencing diminished or inhibited commensal vaginal Lactobacillus species activity and/or expression in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression. In some embodiments, the cerivo-vaginal dysbiosis is related to one or more of Gardnerella vaginalis, Prevotella bivia, and Atopobium vaginae.

In certain embodiments, the present invention provides methods for treating, preventing, and/or reducing dysbiotic vaginal bacteria in a subject through administering to a subject one or more of a donor engraftment of the cervicovaginal mucosa, vaginal microbiota transplant of the cervicovaginal mucosa, and a probiotic colonization of the cervicovaginal mucosa, wherein the donor engraftment, vaginal microbiota transplant, and the probiotic colonization each comprise a therapeutically effective amount of a composition comprising PLA as described herein, wherein the administering is to the cervicovaginal mucosa of the subject. In some embodiments, the administering is a topical administration to the cervicovaginal mucosa of the subject. In some embodiments, the administering is followed by donor engraftment of the cervicovaginal mucosa, vaginal microbiota transplant of the cervicovaginal mucosa, and/or probiotic colonization of the cervicovaginal mucosa. In some embodiments, the subject is experiencing diminished or inhibited commensal vaginal Lactobacillus species activity and/or expression in the subject's microbiome. In some embodiments, the microbiome is a cervico-vaginal microbiome. In some embodiments, the subject is experiencing diminished or inhibited commensal vaginal Lactobacillus species related PLA activity and/or expression. In some embodiments, the subject is experiencing one or more of diminished or inhibited commensal vaginal Lactobacillus crispatus, and/or Lactobacillus paragasseri, and/or Lactobacillus iners activity and/or expression. In some embodiments, the cerivo-vaginal dysbiosis is related to one or more of Gardnerella vaginalis, Prevotella bivia, and Atopobium vaginae.

The PLA may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England (or more recent versions thereof), or (2) direct separation of the mixture of optical enantiomers on chiral chromatographic columns, or (3) fractional recrystallization methods.

The invention further provides processes for preparing any of the compositions of the present invention (see, Example 1).

The invention also provides pharmaceutical compositions comprising PLA in a pharmaceutically acceptable carrier. The invention also provides pharmaceutical compositions comprising PLA associated with either choline or carnitine solubilized (e.g., in water) in a pharmaceutically acceptable carrier.

The invention also provides the uses of such compositions and pharmaceutical compositions.

Such methods described herein are not limited to a particular manner of administering the composition comprising PLA. In some embodiments, the administering is to the cervicovaginal mucosa of the subject. In some embodiments, the administering is a topical administration to the cervicovaginal mucosa of the subject. In some embodiments, such compositions are administered orally (e.g., by oral gavage). Administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, otic, biliary perfusion, cardiac perfusion, priodontal, rectal, spinal subcutaneous, sublingual, topical, intravaginal, transermal, ureteral, or urethral.

The disclosed compositions comprising PLA may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, compositions comprising PLA may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

Compositions comprising PLA may be synthesized according to a variety of methods, including those illustrated in the Examples. Reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4th ed.), John Wiley & Sons, NY (2006), which is incorporated herein by reference in its entirety. Synthesis of the compounds of the disclosure can be accomplished by methods analogous to those described in the synthetic schemes described hereinabove and in specific examples.

When an optically active form of a compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the disclosure as it is defined in the appended claims. All alternatives, modifications, and equivalents of the synthetic methods and specific examples are included within the scope of the claims.

The compositions comprising PLA associated may be incorporated into pharmaceutically acceptable compositions. The pharmaceutical compositions may include a “therapeutically effective amount” or a “prophylactically effective amount” of the compound(s). A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as releasing agents, coating agents, preservatives and antioxidants can also be present in the composition, according to the judgment of the formulator.

The route by which the compositions comprising PLA are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis). In some embodiments, the composition is for oral administration. In some embodiments, the composition is for subcutaneous administration. In some embodiments, the composition is for intravenous administration.

Carriers for systemic administration typically include at least one of diluents, lubricants, binders, disintegrants, antioxidants, preservatives, glidants, solvents, suspending agents, wetting agents, surfactants, cyclodextrins combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, dimethyl sulfoxide, N-methyl-2-pyrrolidone, dimethylacetamide and phosphate (or other suitable buffer). The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about I to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp.⁵87-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Suitable cyclodextrins include alpha-CD, beta-CD, gamma-CD, hydroxypropyl betadex (HP-beta-CD), sulfobutyl-ether β-cyclodextrin (SBE-beta-CD). The amount of cyclodextrins in the systemic or topical composition is typically about 0% to about 40%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and talc.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, preservatives, solvents, suspending agents, and surfactants.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with compositions comprising PLA is sufficient to provide a practical quantity of composition for administration per unit dose of the compound. Techniques and compositions for making dosage forms useful in the methods of this invention are described in the following references: Modem Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

Any of the compositions comprising PLA disclosed herein may further comprise at least one additional therapeutic agent. In some embodiments the at least one additional therapeutic agent. In some embodiments, the at least one additional therapeutic agent comprises any therapeutic for treating conditions associated with diminished or inhibited commensal vaginal Lactobacillus species activity (e.g., sexually transmitted infections (e.g., syphilis, gonorrhoea, chlamydia, trichomoniasis, hepatitis B, herpes simplex virus (HSV), HIV, and human papillomavirus (HPV)), bacterial vaginosis, urinary tract infections).

In another aspect, the disclosure provides kits comprising compositions comprising PLA, and instructions for using the compositions.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed composition and/or product and another agent for delivery to a patient.

The kits can also comprise instructions for using the components of the kit. The instructions are relevant materials or methodologies pertaining to the kit. The materials may include any combination of the following: background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates or flasks).

The kits provided herein are in suitable packaging. Suitable packaging includes, but is not limited to, vials, bottles, jars, flexible packaging, and the like. Individual member components of the kits may be physically packaged together or separately.

### EXAMPLES

The following examples are illustrative, but not limiting, of the compositions and methods of the present invention. Other suitable modifications and adaptations of the variety of conditions and parameters normally encountered in clinical therapy and which are obvious to those skilled in the art are within the spirit and scope of the invention. As used herein, the use of personal pronouns such as “we”, “I”, and/or “our” refers to the inventors.

### Example 1

This example demonstrated that D-PLA as well as L-PLA can be converted to viscous ILs using biocompatible cations.

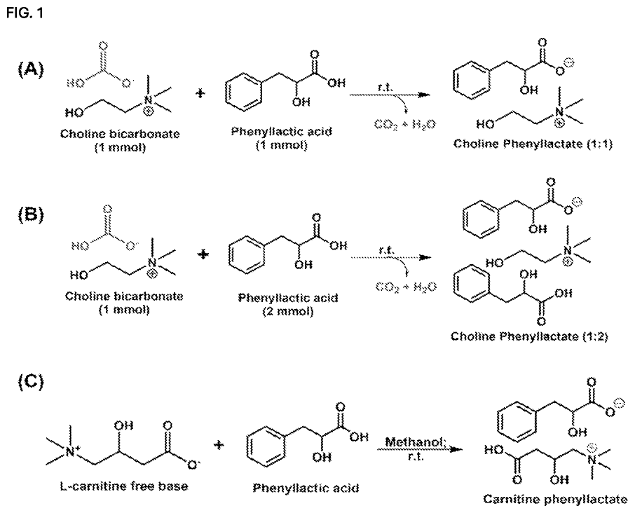

We used previously reported metathesis reaction conditions to enable interaction between choline bicarbonate and D- or L-PLA, at different ratios. As anticipated, choline bicarbonate and D- or L-PLA neutralised each other yielding viscous liquids and carbon dioxide gas as a by-product (Scheme 1A and B; see FIG. 1). After complete drying in a vacuum oven for 48 h, Chol:D-PLA and Chol:L-PLA (both in the ratio of 1:1 and 1:2) remained as clear viscous ILs. The Car:PLA ILs were synthesised by mixing carnitine base and D- or L-PLA in methanol as previously reported for carnitine ILs (Scheme 1C; see FIG. 1). Car:D-PLA and Car:L-PLA were also found to be clear viscous ILs.

### Example 2

This example demonstrated that spectroscopic, chromatographic, and thermal characterization techniques confirmed the formation and purity of PLA ILs.

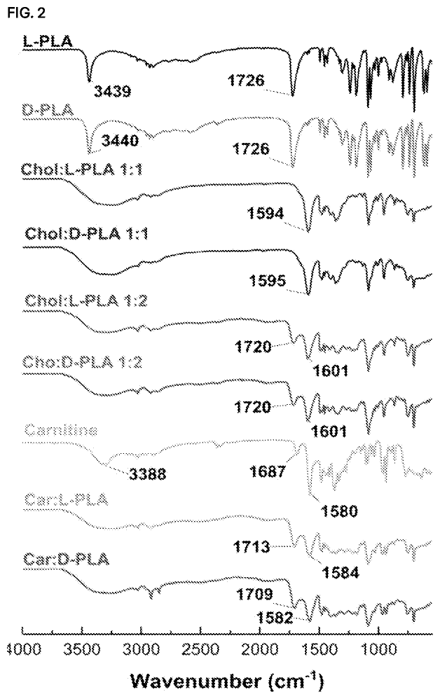

To confirm the formation and purity of PLA ILs we used FT-JR spectroscopy, HPLC, 1H NMR spectroscopy, and thermal techniques. The FT-IR spectrum of D-PLA or L-PLA showed a sharp peak at ~3400 cm-1 and a broad peak at ~2900 cm-1 corresponding to the hydroxyl —OH stretch and carboxylic acid —OH stretch respectively (FIG. 2). The spectra also demonstrated the peak of carboxylic acid (—C═O) stretch at 1726 cm-1. Similarly, the FT-IR spectrum of carnitine showed the hydroxyl stretching and bending at 3388 and 1580 cm-1, while the peak at 1687 cm-1 corresponds to the carboxylic acid (—C═O) stretch. Upon the formation of Chol:D-PLA (1:1) and Chol:L-PLA (1:1) ILs, the carboxylic acid stretch of PLA disappeared, whereas the hydroxyl stretching peak of PLA and choline merged. The new peak at ~1595 cm-1 corresponds to the —OH bending of choline. The FT-IR spectrum of Chol:D-PLA (1:2) and Chol:L-PLA (1:2) showed the peak of carboxylic acid stretch because of an extra mole of PLA but with a significant shift (1720 cm-1). Similarly, carnitine-based PLA ILs demonstrated a significant shift in the peaks corresponding to —C═O stretching and —OH bending (FIG. 2). The FT-IR observations indicate the electrostatic interactions between D- or L-PLA and biocompatible cations.

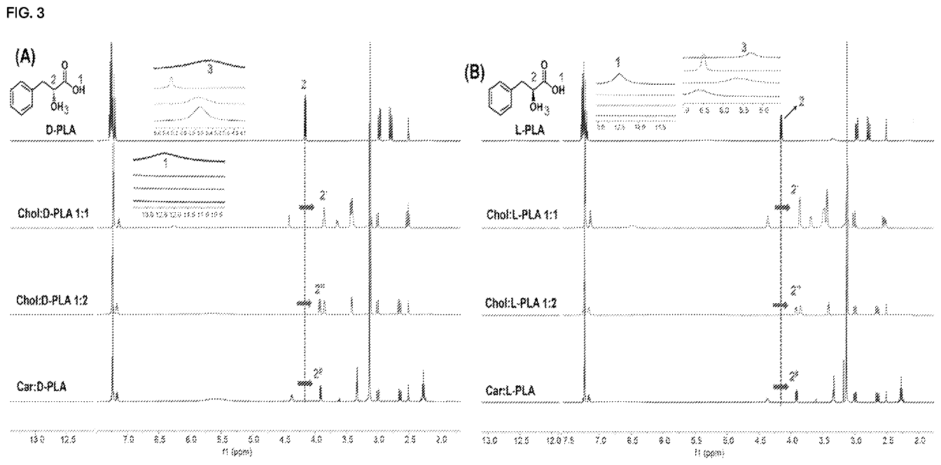

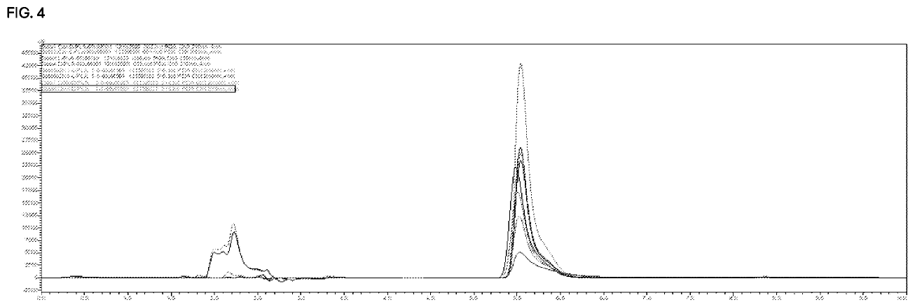

¹H NMR spectrum of D-PLA, L-PLA, Chol:D-PLA, Chol:L-PLA, Car:D-PLA, and Car:L-PLA confirmed the formation of ion pair (FIG. 3). The signal from the —COOH of the D-PLA (12.30 ppm) as well as L-PLA (12.47 ppm) disappeared in the NMR spectra of all PLA ILs (FIGS. 3A and B) confirming the electrostatic interaction between PLA and counter ions (FIGS. 3A and B). The hydroxyl proton shifts of D-PLA and L-PLA (5.30 ppm) showed significant displacement in all PLA ILs. Additionally, the proton adjacent to the hydroxyl group of the PLA (—CH: 2) showed prominent displacement in the NMR spectra of all PLA ILs. This could be due to the hydrogen bonding and steric hindrance of the corresponding counterions. We confirmed the purity of the synthesized PLA ILs using HPLC (FIG. 4).

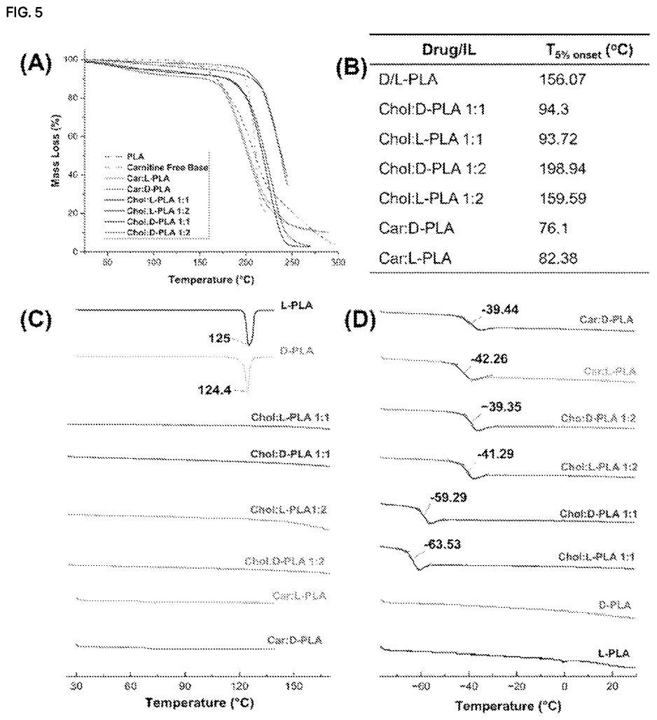

Thermogravimetric analysis (TGA) was used to determine the thermal stability of pure PLA, carnitine, and PLA ILs, and the temperature corresponding to the 5% loss of the total mass of the sample was measured (T_{5%}). The T_{5%} was reached at 156.07° C. for the pure L-PLA compound, while for the pure carnitine free-base, it was at 189.9° C. (FIGS. 5A and B). The T_{5%} for Car:L-PLA (1:1) and Car:D-PLA (1:1) ILs was at 82.38° C. and 76.1° C., respectively. The Chol:D-PLA (1:1) and Chol:L-PLA (1:1) IL T_{5%} were almost similar, 94.3° C. and 93.72° C., respectively. However, T_{5%} values increased to 198.94° C. and 159.59° C. for Chol:D-PLA (1:2) and Chol:L-PLA (1:2) respectively. Thus, carnitine-based PLA (1:1) and choline-based PLA (1:1) ILs showed a decrease in thermal stability while Chol:PLA ILs (1:2) demonstrated improvement.

The differential scanning colorimetric (DSC) analysis of D-PLA, L-PLA, carnitine free base, and all PLA ILs is shown in FIG. 5C. The DSC thermogram of both L- and D-PLA showed sharp endotherm at 125° C. and 124.4° C. respectively, which confirms their crystallinity. The thermograms of carnitine-based ILs and choline-based ILs of PLA did not show the melting peaks of PLA. All PLA ILs showed a slight change in baseline [~70° C. in Car:PLA (1:1), ~130° C. in Chol:PLA (1:2) and ~90° C. in Chol:PLA (1:1)], corroborating the TGA data of these ILs (FIGS. 5A and B). The DSC thermograms of PLA ILs suggest the total distortion of PLA crystalline lattice and conversion into the amorphous form.

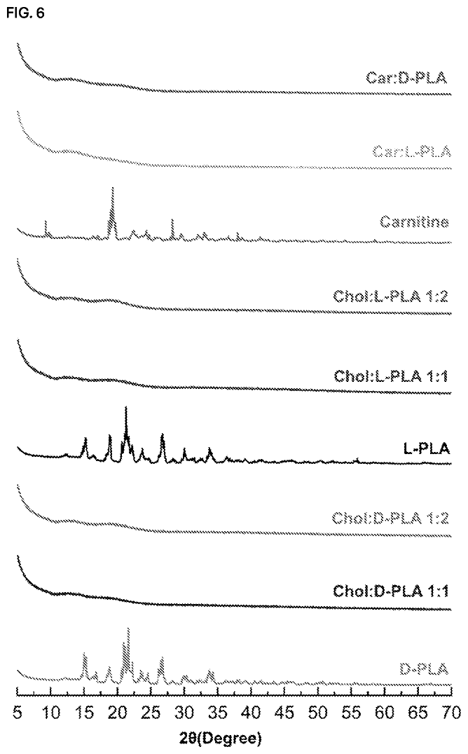

The glass transition (T_{g}) temperatures of Chol:D-PLA (1:1) and Chol:L-PLA (1:1) ILs were −63.53° C. and −59.29° C., respectively (FIG. 5D) whereas the T_{g} temperatures for Chol:D-PLA (1:2) and Chol:L-PLA (1:2) ILs drastically increased to −41.29° C. and −39.35° C., respectively, suggesting the differential amorphicity because of extra moles of PLA in choline-based IL. The T_{g} temperature for Car:D-PLA (1:1) and Car:L-PLA (1:1) compounds were almost similar to that of Chol:D-PLA (1:2) and Chol:L-PLA (1:2) ILs (FIG. 5D). The appearance of the T_{g} curve in all PLA ILs confirms the total amorphization of crystalline PLA. X-ray diffractogram of D- and L-PLA, carnitine, and choline based ILs is shown in FIG. 6. Pure D-PLA showed sharp diffraction peaks at 20 of 15, 18, 21, and 260 and L-PLA displayed peaks at 15, 18, 21, 24, and 26°, confirming their crystallinity. Similarly, L-carnitine demonstrated sharp diffraction peaks at 9, 19, 22, and 28°. The absence of diffraction peaks in the X-ray diffractogram of all PLA ILs (FIG. 6) proves that choline and carnitine successfully converted PLA into amorphous form.

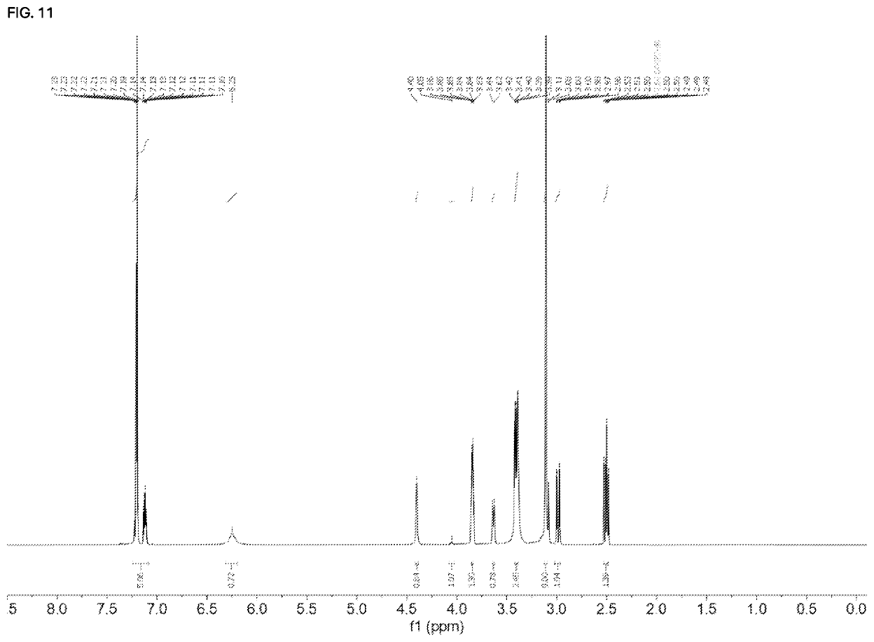

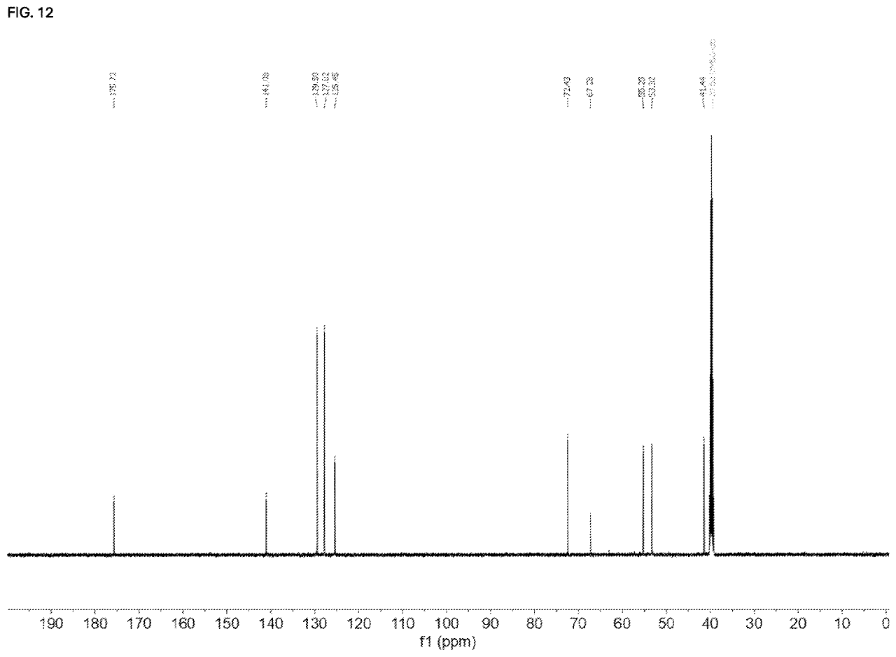

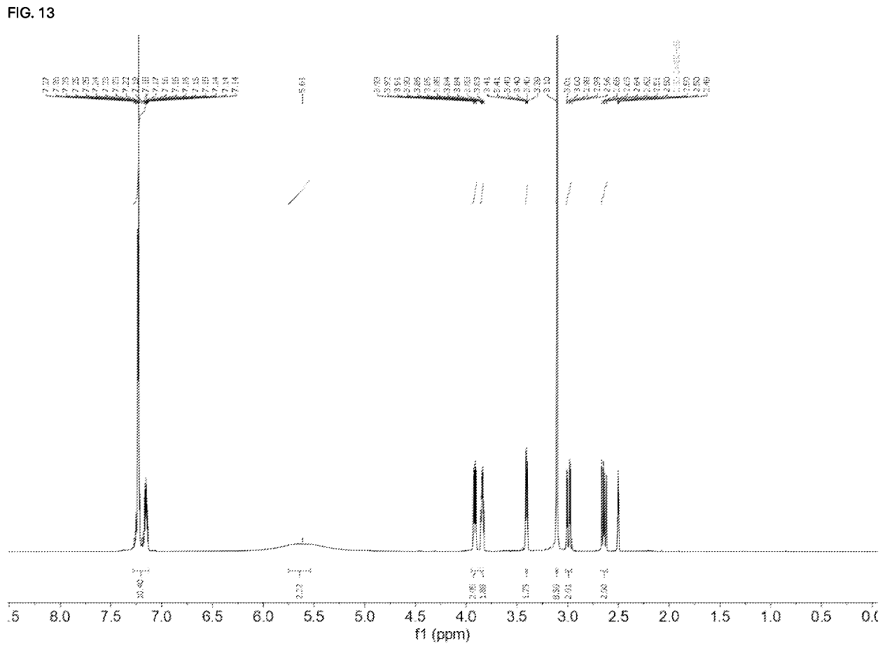

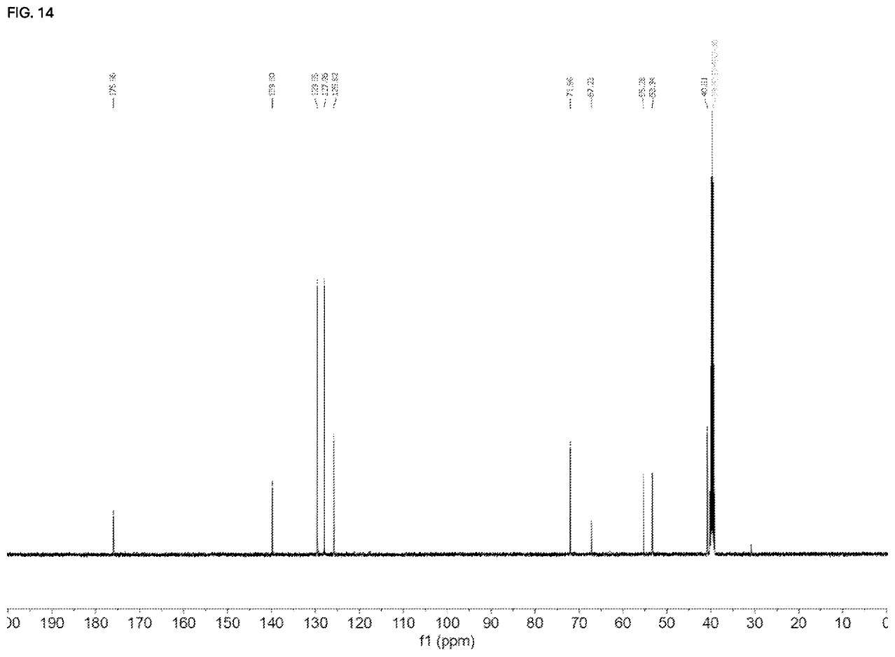

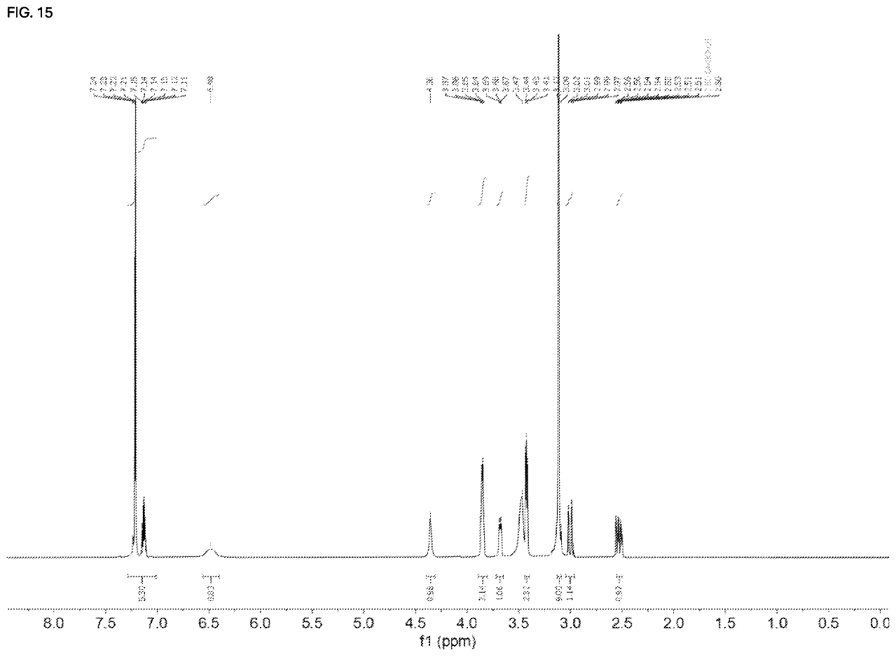

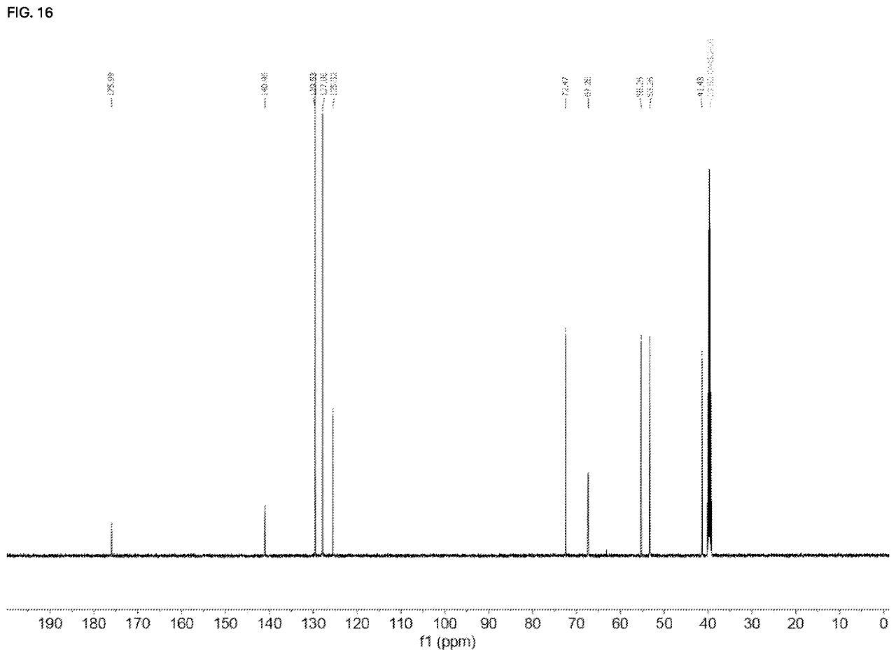

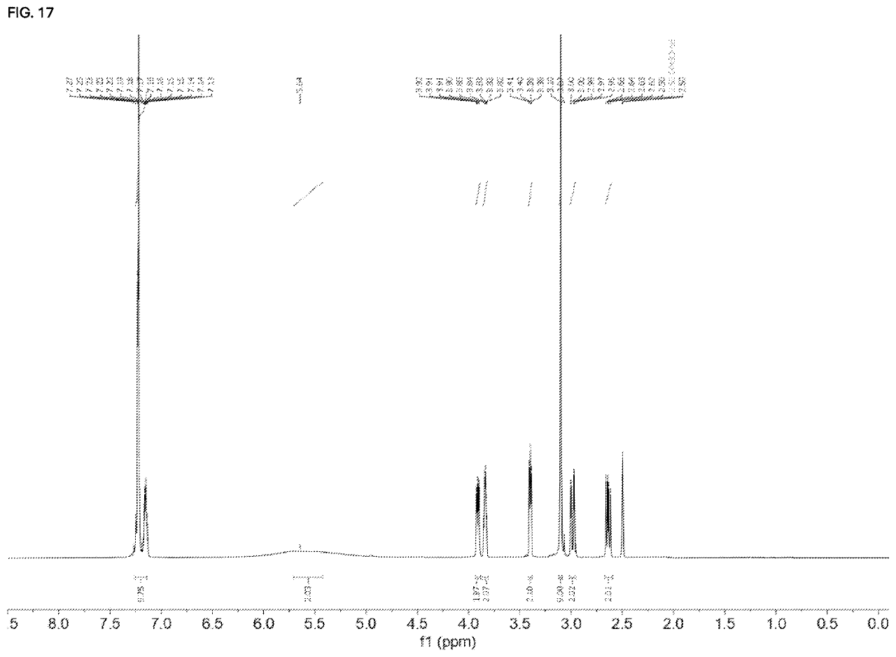

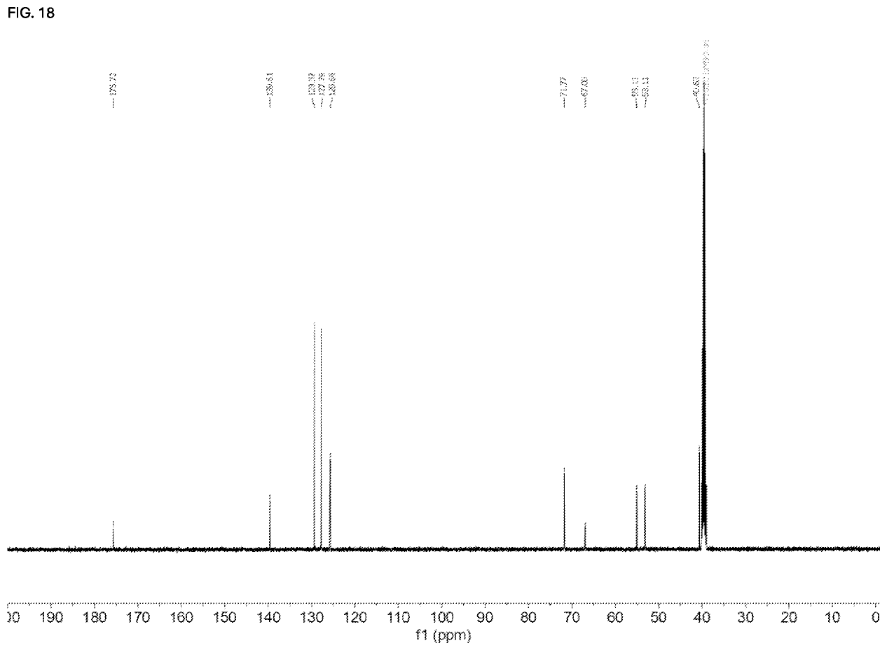

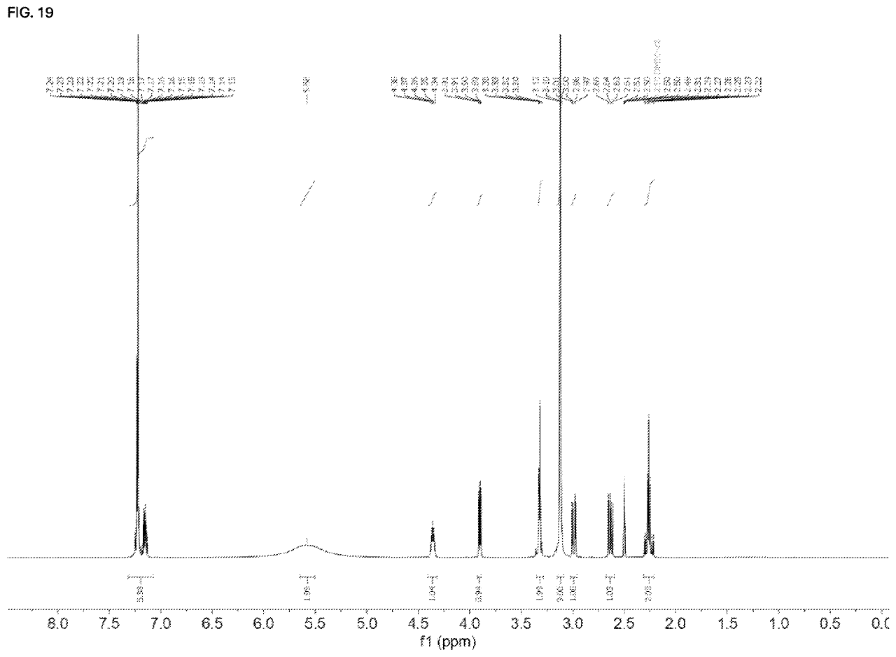

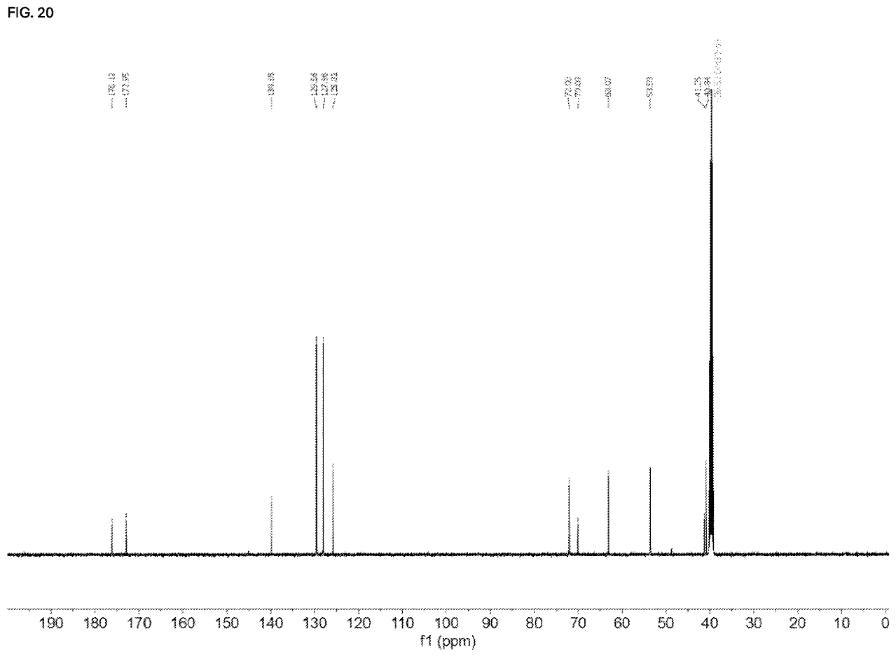

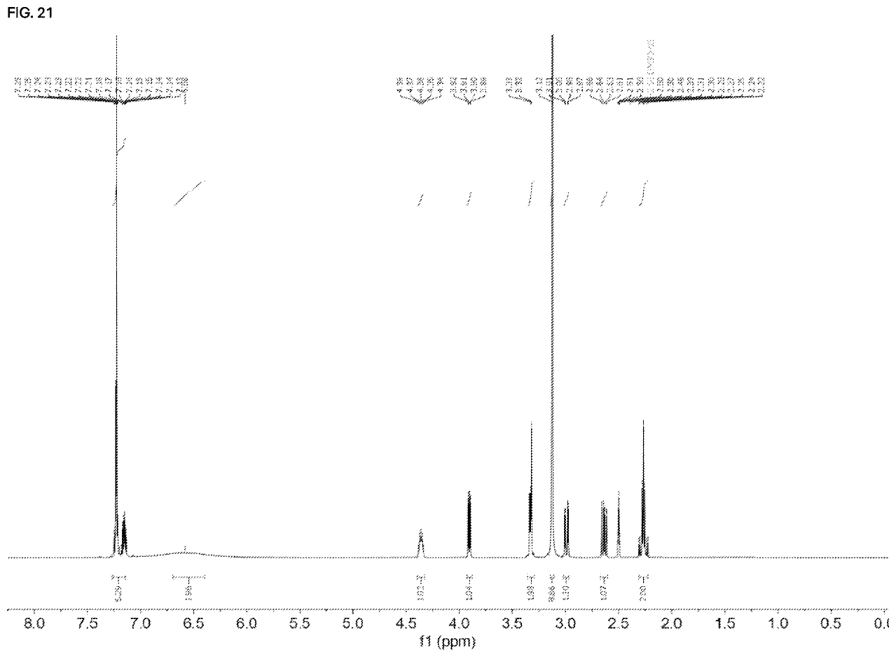

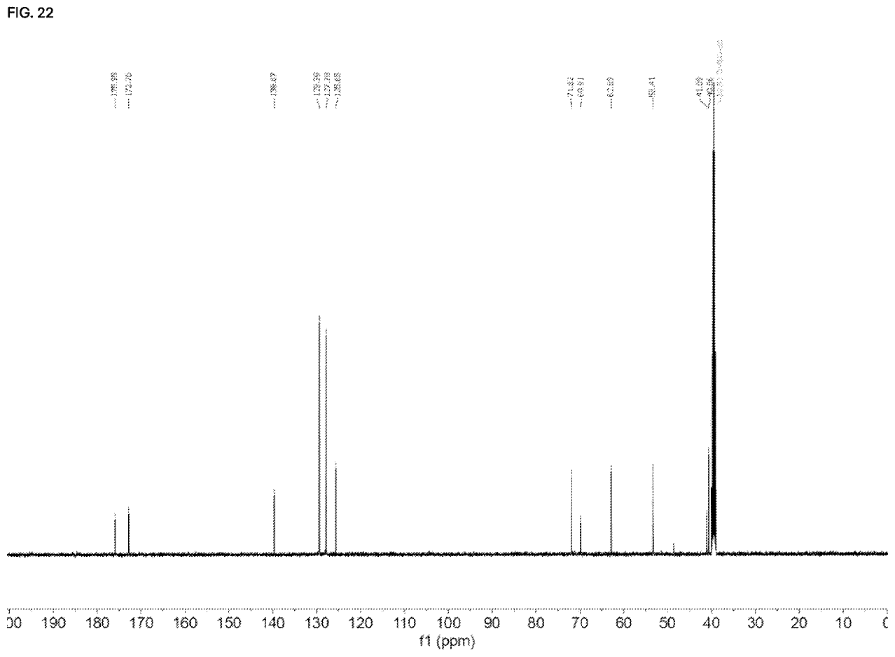

FIGS. 11 through 22 provide ¹H NMR spectrum for various associations of PLA and either choline or carnitine.

### Example 3

This example demonstrates that PLA ILs demonstrate high water solubility.

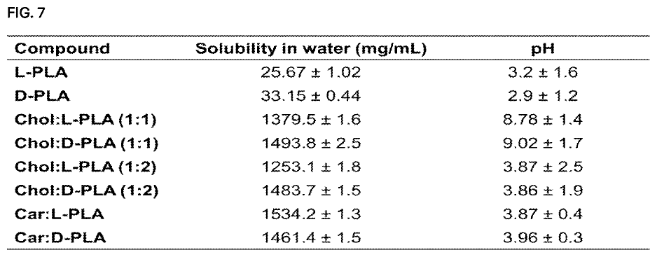

The experimental saturation solubility of D & L-PLA in water was found to be 33.15±0.44 & 25.67±1.02 mg/mL respectively (Table 1 provided in FIG. 7). On the contrary, all PLA ILs showed significantly higher aqueous solubility (Table 1 provided in FIG. 7) irrespective of the cation type, PLA isomer, and the ratio of PLA to cation. As expected, upon formation of an ionic bond with hydrophilic counterion like choline at a 1:1 molar ratio, the solubility of the resulting IL of both the isomers of PLA increased significantly (Table 1). The pH of PLA ILs containing a 1:1 molar ratio of choline and PLA showed alkaline pH (~9) whereas Chol:D-PLA (1:2), Chol:L-PLA (1:2), Car:D-PLA (1:1) and Car:L-PLA (1:1) ILs showed acidic pH (~4).

### Example 4

This example demonstrates that low concentrations of PLA ILs are not cytotoxic to cervical epithelial cells.

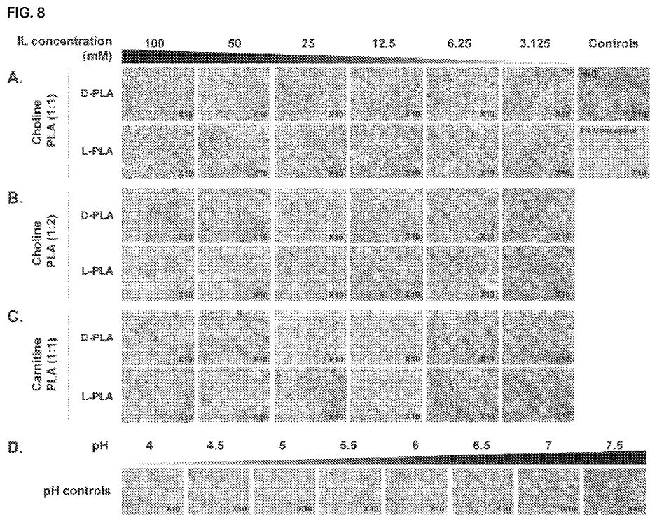

To determine the cytotoxic effects of the synthesised PLA ILs on the cervical epithelium, cervical epithelial cell morphology was visualised by crystal violet staining following treatment with Chol:D-PLA (1:1), Chol:L-PLA (1:1), Chol:D-PLA (1:2), Chol:L-PLA (1:2), Car:D-PLA (1:1), and Car:L-PLA (1:1) ILs. Untreated cells maintained a healthy, confluent monolayer with a healthy ratio of cytoplasm to nuclear cellular content. Following 24 h treatment with Chol:D-PLA (1:1) or Chol:L-PLA (1:1) no morphological changes were observed even at 100 mM (FIG. 8A). However, following treatment with Chol:PLA (1:2) and Carn:PLA (1:1) ILs, decreased cellular density and increased cellular damage was observed at higher concentrations of 100 to 25 mM (FIGS. 8B and C). At lower concentrations of 12.5 to 3.125 mM, no morphological changes were observed, and cell appearance was similar to untreated controls. Car:D-PLA (1:1) and Car:L-PLA (1:1) ILs, however, marginally reduced cell confluency at 12.5 mM compared to Chol:D-PLA (1:2) and Chol:L-PLA (1:2). Cervical epithelial monolayers were also treated with hydrochloric acid (HCl) control solutions with pH ranging from 4 to 7.5 for 24 h and stained with crystal violet to observe the effect of pH on cell morphology. At low pH, similar morphological changes to high concentrations of PLA IL were observed (FIG. 8D). This largely indicates that the observed morphological changes at high PLA IL concentration was likely due to the low pH of the solutions and that, at lower concentrations, PLA ILs cause no morphological changes to cervical epithelial cells.

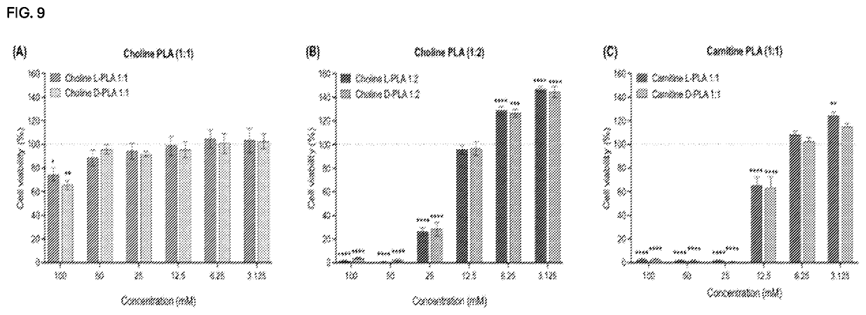

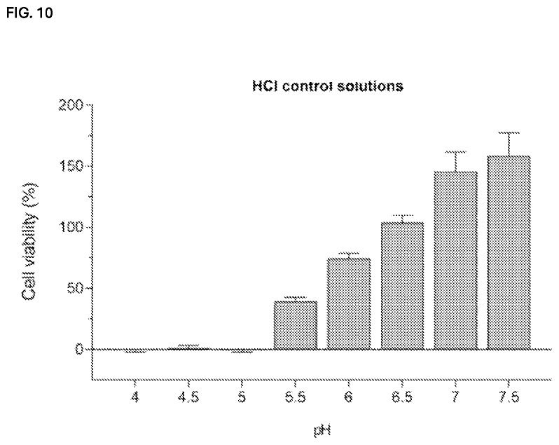

To quantify changes in cell viability following PLA ILs treatment, we conducted MTT assays. MTT is only metabolised by actively proliferating cells, thus is used to measure cellular metabolic activity as an indicator of cell viability. After 24 h, treatment with Chol:D-PLA (1:1) and Chol:L-PLA (1:1) ILs did not significantly reduce cervical epithelial cell viability, even at high concentrations of 50 mM (FIG. 9A). On the other hand, Chol:PLA (1:2) and Car:PLA (1:1) ILs significantly reduced cell viability at concentrations of 100 to 25 mM (P<0.0001) and 100 to 12.5 mM (P<0.0001), respectively (FIGS. 9B and C). Following treatments of 100-50 mM Chol:PLA (1:2) and 100-25 mM Car:PLA (1:1), mean percentage cell viabilities were reduced similarly to between 0.4-4.1% and 0.5%-3.6%, respectively. At 25 mM of Chol:PLA (1:2) and 12.5 mM of Car:PLA (1:1), mean percentage cell viabilities increased to approximately 27.4% and 64.4%, respectively, although were still significantly different from untreated controls (P<0.0001). Interestingly, however, at lower concentrations (6.25 to 3.125 mM) of Chol:D-PLA (1:2) and Chol:L-PLA (1:2) ILs, we observed significantly increased cell viability compared to untreated controls (P<0.001). This trend was also observed at 3.125 mM of carnitine-based PLA ILs, although only significantly for the L-isoform (P<0.01). ILs containing L-PLA overall showed marginally greater toxicity than D-PLA. HCl controls revealed that low pH, equivalent to the pH of Chol:PLA (1:2) and Car:PLA (1:1) ILs at high concentrations, cause similar level of cytotoxicity as corresponding ILs (FIG. 10). These findings corroborate our crystal violet staining observations and further demonstrate that choline-based PLA ILs had the least impact on cervical epithelial cells morphology and cytotoxicity, that low pH of ILs impact cell viability, and that all PLA IL formulations cause no cytotoxicity at lower concentrations.

### Example 5

This example provides a discussion related to Examples 1-4.

A dysbiotic vaginal microbiome, characterised by depletion of the optimal Lactobacillus-dominant community state and overgrowth of diverse anaerobes, can have a severe impact on women's health and lead to increased risk of bacterial vaginosis (BV), sexually transmitted infections (STIs), and pregnancy complications. In turn, these can cause more life-threatening consequences including gynaecological cancers, preterm birth, spontaneous miscarriage, pelvic inflammatory disease, and endometritis.³² Current therapies for modulating the vaginal microbiome largely rely on antibiotics and antifungals to target infections, however, these are often ineffective against recurrent conditions, leading to overuse of antibiotics and contributing to the growing incidence of bacterial resistance. Several novel therapeutic approaches have therefore been proposed with promising results, including probiotics, prebiotics, biofilm disruptive agents, novel antimicrobials, and vaginal microbiota transplant (VMT).³³ We proposed that using metabolites produced by healthy constituents of the vaginal microbiome, such as PLA, would be a promising approach to modulate the vaginal microbiome, aid in restoring homeostasis, and reduce the risk of serious gynaecological and obstetric outcomes. However, these metabolites would need to be soluble and non-toxic to the cervicovaginal epithelium.

PLA has numerous promising antibacterial, antifungal, and immunomodulatory properties but previous studies indicated that a high concentration of PLA is required to elicit these effects. Although the equilibrium solubility of D- and L-PLA in water is >25 mg/ml (Table 1 provided in FIG. 7), the kinetic solubility of D- and L-PLA is considerably lower, and our preliminary studies and previous reports²⁷ show that toxic solvents such as DMSO and ethanol are necessary to solubilize PLA. However, DMSO or ethanol, due to their toxicity to human epithelial cells, would not be appropriate for intravaginal delivery. Hence, a strategy that uses biocompatible ingredients to yield high solubility of PLA is needed to maintain its safety for use on human mucosal surfaces.

In recent years, ionic liquids (ILs) have been extensively researched for their use in biomedicine, due to their ability to solubilise compounds for drug formulations or drug delivery systems. Overall, ILs enable greater solubility, permeability, and bioavailability of compounds. Although previously some ILs have demonstrated toxic properties³⁴, this has been combated through formulating ILs with more biocompatible counterions, namely those of natural source.³⁵ In the past decade, ILs based on endogenous cation, choline, and organic acids have been explored for numerous drug delivery applications due to their excellent solubility, biocompatibility, and permeability across various biological membranes including epithelial surfaces. For example, choline-based ILs were identified to have enhanced solubility and cause lower cytotoxicity to human keratinocyte cells, compared to imidazole-based ILs, amongst the most studied ILs, and were concluded as more suitable for incorporation into topical drug formulations.³⁶ Further, they have been investigated as drug delivery systems for anticancer agents³⁷ and shown to significantly enhance the solubility of ibuprofen³⁸, both without inducing cytotoxicity.

While choline-based biocompatible ILs are being widely explored for drug delivery, other biocompatible cations such as carnitine have been minimally explored for the synthesis of biocompatible ILs. Camitine exists as two biologically active enantiomers, L- and D-carnitine, of which only L-carnitine is endogenously synthesised by the human liver, kidneys, and brain.

Although carnitine-based ILs are less studied, one report showed they can be combined with ascorbic acid and benzoic acid derivatives to enhance solubility in water and form antioxidant ILs^{⇄}. Carnitine-based ILs have also been utilised to synthesise herbicidal ionic liquids with low toxicity and enhanced biodegradability⁴⁰, and to stabilise and solubilise antimicrobial eye drops.⁴¹ Hence, we envisaged that endogenous cations, choline, and L-carnitine could be readily used to transform D- and L-PLA into ILs. Our previous studies show that the biocompatible anion used for the synthesis of ILs impacts the solubility and in vitro cytocompatibility of TLs.^{30, 42} However, the effect of biocompatible cation used for the IL synthesis on the physicochemical properties and cytocompatibility of ILs is not explored. Hence, we focused on the evaluation of the physicochemical properties and cytocompatibility of choline or carnitine-based PLA ILs.

Using previously reported methods, we were able to convert both D- and L-PLA into viscous ILs using equimolar quantities of choline bicarbonate or L-carnitine. The NMR spectra of resulting D- or L-PLA ILs showed the absence of carboxylate proton of PLA and significant shifts in other protons indicating the ionic interaction between D- and L-PLA and cations. We used several other characterization techniques to confirm the formation and purity of PLA ILs. As anticipated, the D- or L-PLA ILs containing 1:1 molar ratio of choline or carnitine showed >50-fold higher aqueous solubility compared to pure D- or L-PLA. However, the pH values of Chol:L-PLA (1:1) and Chol:D-PLA (1:1) were ~9 mainly due to the high basicity of choline (pKa: 11.2). On the contrary, Car:PLA (1:1) ILs showed pH value of ~4 due to its zwitterionic nature and low pKa (3.8) of carnitine. For effective intravaginal delivery, the vaginal formulations should maintain a pH akin to the acidic milieu in the vagina (pH ~4-4.5). Hence, Car:PLA (1:1) ILs were deemed to be suitable for further evaluation. However, to increase the acidity of choline-based PLA ILs for intravaginal applications, we decided to synthesize PLA ILs containing choline and D- or L-PLA at a 1:2 molar ratio. The addition of another mole of D-or L-PLA to choline-based ILs considerably impacted the thermal stability and glass transition temperature of the Chol:PLA (1:2) ILs. Furthermore, the inclusion of an additional mole of PLA in the ILs significantly reduced the pH from ~9 to a pH value of 4 making it suitable for intravaginal application while maintaining the high water solubility of PLA.

The PLA ILs of similar low pH, Car:PLA (1:1) and Cho:PLA (1:2), differed only minimally in their in vitro cytotoxicity analysis, in which slightly higher concentrations of Cho:PLA (1:2) were tolerated by cervical epithelial cells compared to Car:PLA (1:1). This indicates that either choline is a less toxic counterion than carnitine or that a higher ratio of PLA aids in reducing cytotoxicity. ILs with higher pH, Cho:PLA (1:1) ILs, were considerably less cytotoxic than both Cho:PLA (1:2) and Car:PLA (1:1) ILs. This pattern closely corresponded with cytotoxicity observed with hydrochloric acid pH controls, indicating that a more neutral pH results in reduced cytotoxicity, and that the acidic pH of Car:PLA (1:1) and Cho:PLA (1:2) may be the ultimate explanation for their cytotoxicity at high concentrations. Since a lower pH of ILs is preferred for use in the vaginal microenvironment, future formulations of PLA ILs could involve their development into gels to help reduce the impact of pH on the epithelium.

Interestingly, at low concentrations of Cho:PLA (1:2) ILs cell viability was significantly enhanced compared to untreated controls. This was not observed for Cho:PLA (1:1), and only marginally for Car:L-PLA (1:1), further supporting that a greater PLA ratio reduces cytotoxicity and may even enhance epithelial cell growth. Physiological concentrations of lactic acid produced by vaginal Lactobacilli sp. were recently shown to enhance cervicovaginal epithelial barrier integrity by promoting intracellular tight junction protein expression⁴³, and have also demonstrated anti-inflammatory activity.⁴⁴ Although these observations were not specific to aromatic lactic acids or PLA, this provides a potential mechanism in which PLA ILs could enhance the cervicovaginal epithelium as observed in the present study. Future experiments utilising our human three-dimensional (3D) cervical epithelial cell model^{19, 45}, that recapitulates the characteristics of human cervical epithelium, could aid in elucidating the immunomodulatory and barrier-protective role of PLA ILs in the cervicovaginal microenvironment and help identify the safest PLA IL formulation for further development. Use of the 3D model would also enable assessment of the antibacterial, antifungal, and antiviral activities of the PLA ILs following colonisation of the epithelial cells with commensal vaginal microbiota or infection with pathogenic microorganisms, including common vaginal STIs, fungi, and viruses.

Beyond the use of PLA ILs as vaginal microbiome modulators, they could be highly applicable in other areas of biomedical research due to the vast protective and immunomodulatory properties of PLA. In a mouse model, it was found that PLA upregulates intestinal peroxisome proliferator-activated receptor γ (PPAR-γ) activity, a lipid metabolism regulator to protect against metabolic dysfunction, generated by early-life antibiotic exposure and high-fat diet, and ultimately prevent early-life obesity.⁴⁶ Aromatic lactic acids, including PLA, produced by Bifidobacterium species in the infant gut can modulate gut health and immune development.⁴⁷ Similarly, PLA was found to protect against Salmonella enterica serovar Typhimurium-induced colitis in mice by regulating intestinal microbiota, increasing abundance of Lactobacillus, and eliminating inflammation.¹⁸ Furthermore, PLA was previously patented as a skin-protecting ingredient to reduce skin wrinkles¹⁶, highlighting its potential role in the cosmetic industry. When synthesised into a highly soluble, bioavailable IL, these properties of PLA could now be fully exploited for pharmaceutical use.

### Example 6

This example provides the materials and methods pertaining to Examples 1-5.

### Materials

D-PLA and L-PLA were purchased from TCI Chemicals (Portland, OR, USA). Choline bicarbonate was obtained from Sigma Aldrich (St. Louis, MO, USA). L-Carnitine free base was purchased from Spectrum Chemical (Gardena, CA, USA). Methanol (analytical reagent grade) was purchased from VWR International (Radnor, PA, USA). All other chemicals used were of analytical grade unless otherwise indicated.

### High-Pressure Liquid Chromatography Method for PLA

To evaluate the purity of the synthesized D- or L-PLA ILs and for the determination of their water solubility, a reverse phase-HPLC method was developed. The HPLC apparatus consisted of a Shimadzu LC2050C-3D system with a PDA detector. Samples were run through a C₁₈ precolumn and a Gemini C₁₈ reverse-phase column [150×4.5 mm (ID.)] with 5 μm particle size packing (Phenomenex, Torrance, CA). The mobile phase contained acetonitrile and 20 mM phosphoric acid solution in water (25:75). The flow rate of the mobile phase was at 0.8 mL/min, the column oven was set at 45° C., the injection volume was 10 μL, and the detection was carried out at 210 nm. The retention time for PLA was 5.5 min. For the standard curve, D- or L-PLA stock solutions (0.1 mg/mL) were prepared in methanol. The stock solutions were diluted to obtain solutions of various concentrations. The standard curve was obtained by injecting 5-25 μg/mL of PLA. All the experiments were performed in triplicate.

### Synthesis of Chol:D-PLA and Chol:L-PLA ILs

For the transformation of D-PLA and L-PLA into IL, choline bicarbonate (Chol; 80 wt % solution, 0.605 mmol) was added dropwise to the D-PLA and L-PLA (0.605 mmol for 1:1 molar ratio; 1.210 mmol for 1:2 molar ratio) in an open round bottom flask. The mixture was stirred at room temperature until the CO₂ effervescence stopped. After that, the mixture was dried on a rotary evaporator at 60° C. for 1-2 h to remove water followed by drying in a vacuum oven for 2 days to obtain a clear viscous liquid product (yield: 98%).

### Chol:D-PLA (1:1).

¹H NMR (500 MHz, DMSO-d6) δ 7.24-7.08 (m, 5H), 6.25 (s, 1H), 4.40 (s, 1H), 4.05 (s, 1H), 3.85 (d, J=4.6 Hz, 1H), 3.63 (d, J=8.5 Hz, 1H), 3.41 (dd, J=8.6, 3.6 Hz, 2H), 3.11 (s, 9H), 2.99 (dd, J=13.7, 3.1 Hz, 1H), 2.53-2.48 (m, 1H). ¹³C NMR (500 MHz, DMSO-d6) δ 175.72, 141.08, 129.50, 127.82, 125.45, 72.43, 67.28, 55.25, 53.32, 41.44.

### Chol:D-PLA (1:2).

¹H NMR (500 MHz, DMSO-d6) δ 7.28-7.13 (m, 10H), 5.61 (s, 2H), 3.92 (dd, J=8.6, 3.7 Hz, 2H), 3.84 (dq, J=5.0, 2.6 Hz, 2H), 3.42-3.39 (m, 2H), 3.10 (s, 8H), 2.99 (dd, J=13.7, 3.7 Hz, 2H), 2.64 (dd, J=13.7, 8.6 Hz, 2H). ¹³C NMR (500 MHz, DMSO-d6) δ 175.96, 139.80, 129.55, 127.96, 125.82, 71.96, 67.23, 55.28, 53.34, 40.81.

### Chol:L-PLA (1:1).

¹H NMR (500 MHz, DMSO-d6) δ 7.28-7.01 (m, 5H), 6.48 (s, 1H), 4.36 (s, 1H), 3.85 (q, J=5.4 Hz, 2H), 3.72-3.65 (m, 1H), 3.44-3.40 (m, 2H), 3.12 (s, 9H), 3.00 (dd, J=13.7, 3.1 Hz, 1H), 2.55-2.50 (m, 1H). ¹³C NMR (500 MHz, DMSO-d6) δ 175.99, 140.96, 129.53, 127.86, 125.52, 72.47, 67.28, 55.26, 53.26, 41.43.

### Chol:L-PLA (1:2).

¹H NMR (500 MHz, DMSO-d6) δ 7.25-7.14 (m, 1OH), 5.64 (s, 2H), 3.91 (dd, J=8.5, 3.6 Hz, 2H), 3.86-3.81 (m, 2H), 3.40 (dd, J=6.7, 3.4 Hz, 2H), 3.10 (s, 9H), 2.99 (dd, J=13.7, 3.6 Hz, 2H), 2.64 (dd, J=13.7, 8.6 Hz, 2H). ¹³C NMR (500 MHz, DMSO-d6) δ 175.72, 139.61, 129.37, 127.79, 125.66, 71.77, 67.00, 55.11, 53.11, 40.62.

### Synthesis of Car:D-PLA and Car:L-PLA ILs

An equimolar quantity (0.620 mmol) of L-carnitine (Car) and D-PLA or L-PLA were dissolved in 10 mL of methanol. The mixture was stirred on a magnetic stirrer for 3 h at room temperature. After that, the mixture was concentrated on a rotary evaporator to obtain clear viscous liquid (yield: 99%).

### Car:D-PLA.

¹H NMR (500 MHz, DMSO-d6) δ 7.31-7.07 (m, 5H), 5.58 (s, 2H), 4.36 (p, J=6.4 Hz, 1H), 3.90 (dd, J=8.5, 3.7 Hz, 1H), 3.35-3.29 (m, 2H), 3.12 (s, 9H), 2.99 (dd, J=13.7, 3.6 Hz, 1H), 2.63 (dd, J=13.7, 8.6 Hz, 1H), 2.31-2.21 (m, 2H). ¹³C NMR (500 MHz, DMSO-d6) δ 176.12, 172.95, 139.85, 129.56, 127.96, 125.82, 72.00, 70.09, 63.07, 53.59, 41.25, 40.84.

### Car:L-PLA.

¹H NMR (500 MHz, DMSO-d6) δ 7.27-7.14 (m, 5H), 6.58 (s, 2H), 4.36 (p, J=6.8 Hz, 1H), 3.91 (dd, J=8.6, 3.7 Hz, 1H), 3.33 (d, J=3.7 Hz, 2H), 3.12 (s, 9H), 2.99 (dd, J=13.7, 3.6 Hz, 1H), 2.64 (dd, J=13.7, 8.6 Hz, 1H), 2.31-2.22 (m, 2H). ¹³C NMR (500 MHz, DMSO-d6) δ 175.95, 172.76, 139.67, 129.39, 127.79, 125.65, 71.82, 69.91, 62.89, 53.41, 41.09, 40.66

### Fourier-Transform Infrared (FT-IR) Spectroscopy

The FTIR spectroscopy measurements were conducted on a Nicolet iS10 Fourier-Transform Infrared (FTIR) Spectrophotometer (Thermo Scientific, Waltham, MA) with a diamond attenuated total reflection (ATR) unit. FTIR spectra were obtained in ‘% transmission’ mode from 4000 to 500 cm⁻¹ with an average of 16 scans for obtaining background.

### Nuclear Magnetic Resonance (NMR) Spectroscopy

NMR spectra were obtained using a Bruker Avance Digital 500 MHz NMR spectrometer (Bruker Corp., Billerica, MA) and an automatic sample changer, the BACS1. A 5 mm PABBO BB-1H/D Z-GRD probe was mounted to the spectrometer. The ¹³C spectra of each sample were recorded using an average of 1000 scans, and the ¹H spectrum of the refined products (6-8 mg) was obtained using an average of 16 scans in dimethyl sulfoxide-d6 (Cambridge Isotope Laboratories, Inc., Tewksbury, MA). Chemical changes are reported in parts per million (ppm) units.

### Thermogravimetric Analysis (TGA)

TA Instruments Discovery TGA 5500 (TA instruments, New Castle, DE, USA) was used to analyse pure PLA, carnitine, and various PLA ILs. The samples were accurately weighed (2-5 mg) in platinum pans and placed onto the autosampler. The samples were heated from 30° C. to 350° C. at 10° C./min; nitrogen was purged at a rate of 25 mL/min. TRIOS software (TA instruments, New Castle, DE, USA) was used to analyse the data.

### Differential Scanning Calorimetry (DSC)

The TA Instruments DSC 2500 RCS40 cooling unit (TA instruments, New Castle, DE, USA), calibrated with indium, was used to record thermograms of pure compounds of D-PLA, L-PLA, carnitine, and their corresponding ILs. The samples were weighed (6-10 mg) and hermetically sealed in T_{zero} aluminium pans, along with an empty reference T_{zero} pan. The atmosphere was inert because of nitrogen purge at a rate of 25 mL/min. The samples were heated from 30° C. to the temperature equivalent to a 5% weight loss point with a 5° C./min ramp for each sample to observe thermal transitions. The glass transition temperature (T_{g}) for each sample was recorded in the cool/heat run at 5° C./min ramp from 30° C. to −80° C., and from −80° C. to 30° C. TRIOS software (TA instruments, New Castle, DE, USA) was used to analyse the data.

### Powder X-Ray Diffraction (PXRD) Spectroscopy

The crystallinity of samples was assessed using a Philips PANalytical X'Pert PRO MPD (Malvern Panalytical, Malvern, UK) with a copper X-ray source (Kα radiation with λ=1.5406 Å). The PLA, carnitine, or PLA ILs were placed into a 20×20 mm sample holder pocket that was 0.2 mm deep and the surface was leveled. The diffraction pattern of the samples was acquired at room temperature by scanning across a range of 5.0 to 70.0° (2θ) at a speed of 0.63° per second.

### Equilibrium Solubility and pH Determination Studies

The equilibrium solubility of D-PLA, L-PLA, and their ILs was determined using a previously reported method.³⁰ An excess amount of D-PLA, L-PLA, or their ILs was added in an Eppendorf tube along with 1 mL of water. The sample was placed on an orbital shaker and was equilibrated at room temperature for 24 h with a speed of 200 rpm. After 24 h, the samples were centrifuged, the supernatant was filtered using a 0.22 μm syringe filter and further diluted using methanol as necessary. The D-PLA or L-PLA content in the supernatant was analyzed using the HPLC method to calculate the equilibrium solubility. Further, the pH values of D-PLA, L-PLA, and their ILs dissolved in water were determined using a VWR 1100L pH meter.

### Human Endocervical Epithelial Cell Culture

Human endocervical epithelial cell line (A2EN), generated from human endocervical explant tissue³¹, was cultured as monolayers in keratinocyte serum-free medium (KFSM) supplemented with recombinant epidermal growth factor (5 ng/ml), bovine pituitary extract (50 μg/ml; Gibco), CaCl₂ (22 mg/ml; Sigma-Aldrich), and primocin (100 μg/ml; InvivoGen, San Diego, CA), herein referred to as A2 medium, at 37° C. under 5% carbon dioxide (CO₂) atmosphere.

### Preparation of PLA ILs and pH Solutions

Prior to cytotoxicity testing, PLA ILs were diluted to 1 M concentrations with distilled water and filtered using a sterile 0.2 μm syringe filter. Solutions with pH ranging from 4 to 7.5 were prepared by adjusting the pH of A2 media with hydrochloric acid (HCl), following filtration using a sterile 0.2 μm syringe filter.

### Crystal Violet Staining for Cervical Cell Morphology

Cervical cell morphology following PLA IL treatment was visualised using 2.3% crystal violet stain with 0.1% ammonium oxalate and 20% ethanol (Sigma-Aldrich). Cervical cell monolayers were seeded into a tissue-treated 24-well microtiter plate (Falcon) at a density of 1.5×10⁵ cells/well and incubated for 24 h at 37° C. under 5% CO₂. Two-fold serial dilutions of each PLA IL were prepared and aliquoted into respective wells to obtain final concentrations of 3.125 to 100 mM. Deionised water and 1% Conceptrol (v/v), containing the detergent, nonoxynol-9, were used as negative and positive controls, respectively. Following 24 h incubation at 37° C. under 5% CO₂ atmosphere, adhered cells were stained with 100 μL of crystal violet for 5 minutes, washed thoroughly with deionised water until clear, and imaged at 10× magnitude using a bright-field microscope (Olympus IX71). HCl solutions (with pH ranging from 4 to 7.5) were also tested for cell morphology changes following the same protocol.

### MTT Assay for Cervical Cell Proliferation

Cervical cell monolayers were seeded into a tissue-treated 96-well microtiter plate (Corning Costar) at a density of 2.0×10⁴ cells/well and incubated for 24 h at 37° C. under 5% CO₂. Cells were treated with each PLA IL at concentrations ranging to 3.125 to 100 mM and incubated for 24 h (37° C. under 5% CO₂). MTT [3-(4,5-dimethylthiazol-2-yl)-2,5-diphenyltetrazolium bromide](AMRESCO) solution was added to wells at a final concentration of 0.45 mg/ml per the manufacturer's protocol. Absorbance was measured at 570 nm (reference 650 nm) and recorded using the Safire II Multi-Mode Microplate reader (Tecan, Mannedorf, Switzerland). Percentage cell viability was calculated as [sample absorbance−media background absorbance]/[control absorbance−media background absorbance]×100. At least three independent biological replicates were performed per PLA IL. HCl solutions (pH 4-7.5) were also tested following the same protocol.

### Statistical Analysis

All statistical analyses were performed using Prism 9.0 software (GraphPad, San Diego, CA). Two-way ANOVA with Bonferroni's adjustment was used to compare MTT data between PLA ILs and untreated controls. The analysis was conducted on at least three independent biological replicates and displayed as mean with standard error of the mean (SEM). P values <0.05 were considered statistically significant.

### Example 7

Experiments were conducted to investigate the antimicrobial properties of PLA derivatives against commensal and BV-associated bacteria, and its cytotoxic properties on human cervical epithelial cells.

PLA ionic liquids (ILs), organic salts that are liquid at room temperature, were synthesized using choline and carnitine counterions to enable solubilization of D- and L-enantiomers of PLA in water. Disk diffusion and minimum inhibitory concentration (MIC) assays were conducted to determine the antimicrobial activity of the PLA ILs. Two commensal vaginal species (Lactobacillus crispatus, Lactobacillus iners) and three common BV-associated species (Fannyhessea vaginae, Prevotella bivia, Gardnerella vaginalis) were tested. Cytotoxicity of PLA ILs to human cervical epithelial cell line (A2EN) was visualized by crystal violet staining and quantified using MTT assays. Compared to negative controls, 1M concentrations of carnitine and choline D-PLA and carnitine L-PLA significantly inhibited the growth of both commensal and BV-associated bacteria, following 24 hr bacterial growth (P<0.0001). Carnitine D-PLA exhibited the strongest antimicrobial activity, whilst choline L-PLA was the least effective; quantitative MIC assays are on-going to determine inhibitory concentrations for each bacterial species. Cell viability assays revealed carnitine-PLA ILs as more cytotoxic to human cervical cells than choline-PLA ILs with toxicity at 6.25 mM vs. 12.5 mM. However, acidic pH (approx. 5.5) of PLA-ILs could be responsible for the cytotoxicity observed at higher PLA concentrations. Microscopic visualization of cell morphology using crystal violet staining confirmed these results. Here, we found that both the stereoisomerism of PLA and the counterion present (carnitine or choline) altered the antimicrobial activity of the ILs, whilst the level of cytotoxicity was predominately impacted by the counterion present. On-going studies are aimed at determining inhibitory concentrations and testing additional formulations of PLA for antimicrobial activity to better understand its mechanism and potential therapeutic capabilities within the cervicovaginal microenvironment.

PLA ionic liquids (ILs), organic salts that are liquid at room temperature, were synthesized using cations (compound #1 and #2) to enable solubilization of D- and L-enantiomers of PLA in water. Disk diffusion and minimum inhibitory concentration (MIC) assays were conducted to determine the antimicrobial activity of the PLA-ILs against commensal and BV-associated species (see, Table 2). Cytotoxicity of PLA ILs to human cervical epithelial cell line was visualized by crystal violet staining and quantified using MTT assays.

TABLE 2

| | Associated | | |
|---|---|---|---|
| Bacterial strain | with | Characteristics | Source |
| Lactobacillus | Health | Isolated from vagina of | BEI- |
| crispatus JV-V01 | | a healthy woman | RRP |
| Gardnerella | Dysbiosis | Isolated from vagina of | BEI- |
| vaginalis JCP | | a woman tested | RRP |
| 8151B | | positive for BV | |
| Prevotella bivia | Dysbiosis | Type strain; isolated | ATCC |
| VPI 6822 | | from endometrium | |
| Atopobium vaginae | Dysbiosis | Type strain; isolated | ATCC |
| CCUG 38953 | | from vagina of a | |
| | | healthy woman | |

ATCC: American Type Culture Collection
BEI-RRP: Biodefense and Emerging Infectious Research Resources Program (NIH)

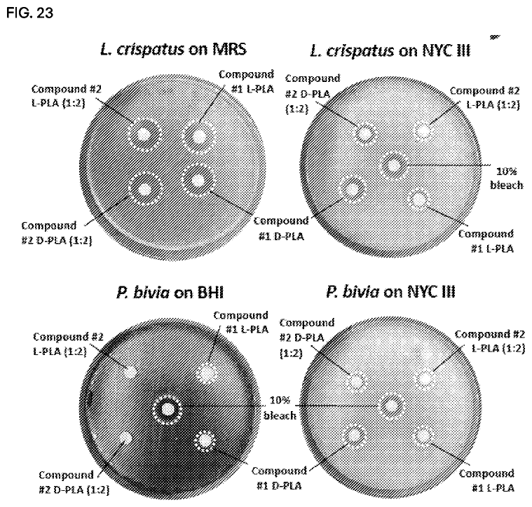

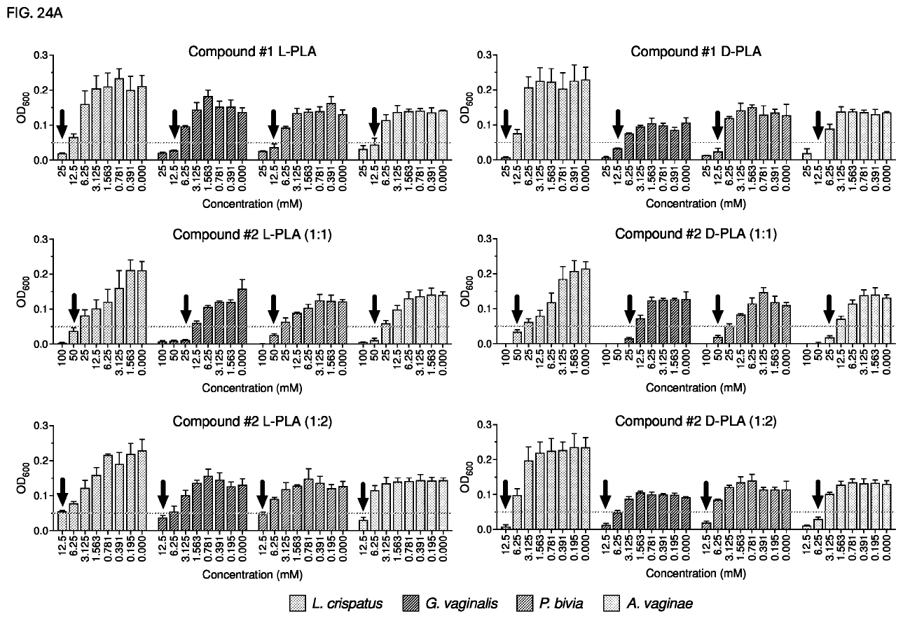

As shown in FIG. 23, PLA-ILs inhibit growth of vaginal bacteria differently based on the agar medium, the IL cation, and the PLA enantiomer. The zone of inhibition of L. crispatus and P. bivia on both their optimum medium and NYC III medium, was measured following treatment with disks immersed in 1M PLA-IL. 10% bleach was used as a positive control. Plates were incubated for 24 hrs at 37° C. in anaerobic conditions.

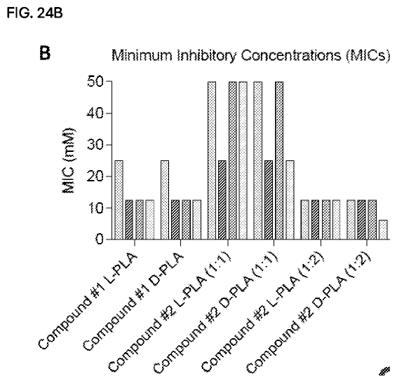

As shown in FIG. 24, the growth of health- and BV-associated bacteria are inhibited more strongly by compounds #1 and #2 PLA (1:2) than by compound #2 PLA (1:1). (A) The MIC for each PLA-IL to inhibit the growth of vaginal bacteria in NYC III broth was measured. Each PLA-IL was serially diluted in the broth and inoculated with bacterial suspension adjusted to OD₆₀₀ of 0.05 (grey dashed line). The OD₆₀₀ was then measured 22 hours post bacterial inoculation. Black arrows indicate MIC. Data plotted as mean+SEM, with 2-3 replicates per compound. (B) Bar graph of the MICs for each compound.

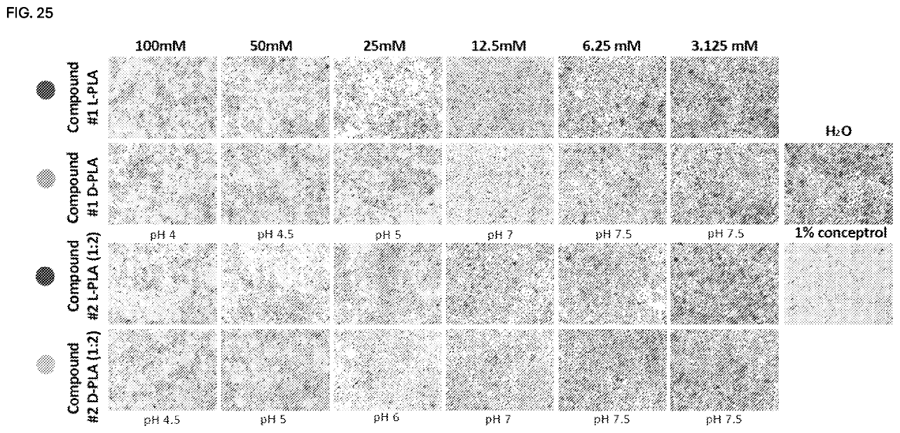

As shown in FIG. 25, Compound #1 PLA does not cause any morphological changes to human cervical epithelial cells at concentrations ≤12.5 mM, and compound #2 PLA (1:2) at 25 mM. Microscopy images, at ×10 magnification, of crystal violet stained cervical epithelial cells, grown as monolayers, following treatment with each PLA-IL. pH values of the cell supernatant shown under the images. 1% conceptrol and H₂O were used as positive and negative controls, respectively. At high PLA-IL concentrations, either the PLA-IL or low pH may have caused cell stress and death.

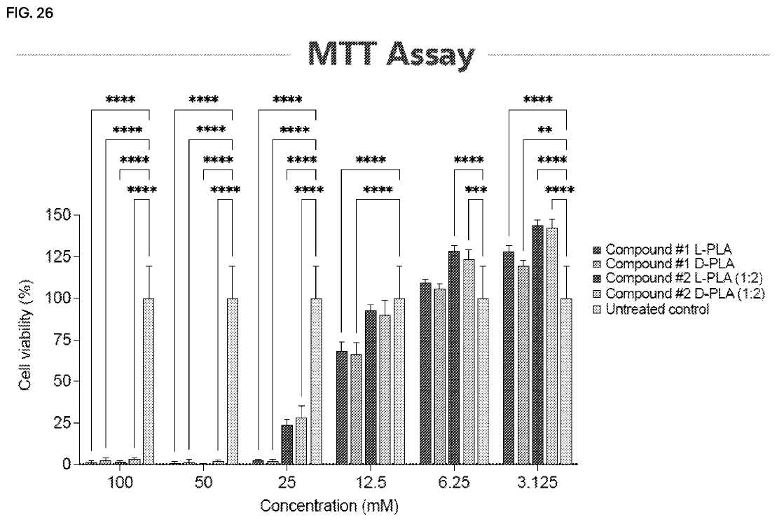

As shown in FIG. 26, Compound #1 PLA does not cause any cytotoxicity to human cervical epithelial cells at concentrations ≤6.25 mM, and compound #2 PLA (1:2) at 12.5 mM. Cervical epithelial cells were treated with increasing concentrations of each PLA-IL, incubated for 24 hrs, and then the MTT assay was conducted. Absorbance was measured at 570 nm and subtracted from 650 nm reference. Percentage cell viability was calculated and plotted as mean+SEM, with 9 replicates per condition. Significance was determined using two-way ANOVA with Bonferroni's adjustments. Asterisks indicate significance (****P<0.0001; ***P<0.001; **P<0.01; *P<0.05).

These results demonstrate, for example, that 1) PLA-ILs have antimicrobial properties against vaginal bacteria and are not cytotoxic to human cervical epithelial cells; 2) Growth of both health-associated Lactobacillus crispatus and BV-associated bacteria are inhibited by PLA-ILs; 3) Compounds #1 and #2 PLA (1:2) exhibit stronger antimicrobial activity than compound #2 PLA (1:1); 4) Compound #1 PLA had stronger antimicrobial properties against BV-associated bacteria then L. crispatus; 5) Compound #1 PLA is more cytotoxic to human cervical cells than Compound #2 PLA (1:2) with toxicity at 6.25 mM vs. 12.5 mM; and 6) Acidic pH (5.5) of PLA-ILs could be responsible for the cytotoxicity observed at higher PLA-IL concentrations.

### INCORPORATION BY REFERENCE

The entire disclosure of each of the patent documents and scientific articles referred to herein is incorporated by reference for all purposes. The following references are herein incorporated by reference in their entireties:
1. W. Mu, S. Yu, L. Zhu, T. Zhang and B. Jiang, Applied Microbiology and Biotechnology, 2012, 95, 1155-1163.
2. E. Dempsey and S. C. Corr, Frontiers in immunology, 2022, 13, 840245.
3. M. L. Neugent, N. V. Hulyalkar, V. H. Nguyen, P. E. Zimmern and N. J. De Nisco, MBio, 2020, 11, 10.1128/mbio. 00218-00220.
4. M. A. Antonio, S. E. Hawes and S. L. Hillier, Journal of Infectious Diseases, 1999, 180, 1950-1956.
5. R. Rajanikar, B. H. Nataraj, H. Naithani, S. A. Ali, N. R. Panjagari and P. V. Behare, Food Control, 2021, 128, 108184.
6. S. Siedler, R. Balti and A. R. Neves, Current opinion in biotechnology, 2019, 56, 138-146.
7. V. Dieuleveux, S. Lemarinier and M. Gueguen, International Journal of Food Microbiology, 1998, 40, 177-183.
8. F. Valerio, P. Lavermicocca, M. Pascale and A. Visconti, FEMS microbiology letters, 2004, 233, 289-295.
9. Y. Ning, Y. Fu, L. Hou, M. Ma, Z. Wang, X. Li and Y. Jia, Food Research International, 2021, 139, 109562.
10. Y.-H. Jiang, J.-P. Ying, W.-G. Xin, L.-Y. Yang, X.-Z. Li and Q.-L. Zhang, Journal of Dairy Science, 2022, 105, 9463-9475.
11. P. Lavermicocca, F. Valerio, A. Evidente, S. Lazzaroni, A. Corsetti and M. Gobbetti, Applied and Environmental Microbiology, 2000, 66, 4084-4090.
12. S. M. Schwenninger, C. Lacroix, S. Truttmann, C. Jans, C. Spörndli, L. Bigler and L. Meile, Journal of food protection, 2008, 71, 2481-2487.
13. V. Dieuleveux, D. Van Der Pyl, J. Chataud and M. Gueguen, Applied and environmental microbiology, 1998, 64, 800-803.
14. J. Wang, J. Yoo, J. Lee, T. Zhou, H. Jang, H. Kim and I. Kim, Journal of Applied Poultry Research, 2009, 18, 203-209.
15. F. J. Urban and B. S. Moore, Journal of heterocyclic chemistry, 1992, 29, 431-438.
16. J. Y. Ruey and E. J. Van Scott, Journal, 1997.
17. A. Peters, P. Krumbholz, E. Jäger, A. Heintz-Buschart, M. V. Çakir, S. Rothemund, A. Gaudl, U. Ceglarek, T. Schöneberg and C. Stäubert, PLoS genetics, 2019, 15, e1008145.
18. Q. Zhou, R. Gu, B. Xue, P. Li and Q. Gu, Food & Function, 2021, 12, 5591-5606.
19. P. Łaniewski and M. M. Herbst-Kralovetz, npj Biofilms and Microbiomes, 2021, 7, 88.
20. N. R. Jimenez, J. D. Maarsingh, P. Łaniewski and M. M. Herbst-Kralovetz, Msphere, 2023, 8, e00452-00422.
21. J. H. Saigh, C. C. Sanders and W. E. Sanders, Infection and Immunity, 1978, 19, 704-710.
22. Z. Gong, Y. Luna, P. Yu and H. Fan, PloS one, 2014, 9, e107758.
23. K. Gupta, A. E. Stapleton, T. M. Hooton, P. L. Roberts, C. L. Fennell and W. E. Stamm, Journal of infectious diseases, 1998, 178, 446-450.
24. S. L. Hillier, M. A. Krohn, L. K. Rabe, S. J. Klebanoff and D. A. Eschenbach, Clinical Infectious Diseases, 1993, 16, S273-S281.
25. M. Falagas, G. Betsi and S. Athanasiou, Clinical microbiology and infection, 2007, 13, 657-664.
26. G. Tachedjian, M. Aldunate, C. S. Bradshaw and R. A. Cone, Research in Microbiology, 2017, 168, 782-792.
27. O. Abdul-Rahim, Q. Wu, T. K. Price, G. Pistone, K. Diebel, T. S. Bugni and A. J. Wolfe, Journal of Bacteriology, 2021, 203, 10.1128/jb.00360-00321.
28. M. K. Shukla, H. Tiwari, R. Verma, W.-L. Dong, S. Azizov, B. Kumar, S. Pandey and D. Kumar, Pharmaceutics, 2023, 15, 702.
29. W. Huang, X. Wu, J. Qi, Q. Zhu, W. Wu, Y. Lu and Z. Chen, Drug Discovery Today, 2020, 25, 901-908.
30. Y. Sutar, S. K. Singh, S. Dhoble, J. Mali, J. Adams, T. Yadavalli, A. A. Date and D. Shukla, ACS Infectious Diseases, 2024, 10, 93-106.
31. M. M. Herbst-Kralovetz, A. J. Quayle, M. Ficarra, S. Greene, W. A. Rose, 2nd, R. Chesson, R. A. Spagnuolo and R. B. Pyles, Am J Reprod Immunol, 2008, 59, 212-224.
32. D. H. Martin and J. M. Marrazzo, The Journal of infectious diseases, 2016, 214, S36-S41.
33. P. Łaniewski, Z. E. Ilhan and M. M. Herbst-Kralovetz, Nature Reviews Urology, 2020, 17, 232-250.
34. J. Flieger and M. Flieger, Int J Mol Sci, 2020, 21.
35. R. Md Moshikur, M. R. Chowdhury, M. Moniruzzaman and M. Goto, Green Chemistry, 2020, 22, 8116-8139.
36. T. Santos de Almeida, A. Júlio, N. Saraiva, A. S. Fernandes, M. E. M. Araújo, A. R. Baby, C. Rosado and J. P. Mota, Drug Development and Industrial Pharmacy, 2017, 43, 1858-1865.
37. R. Caparica, A. Jdlio, M. E. Araújo, A. R. Baby, P. Fonte, J. G. Costa and T. Santos de Almeida, Journal, 2020, 10.
38. J. Yuan, J. Wu and T. Yin, Journal of Drug Delivery Science and Technology, 2020, 60, 102037.
39. K. Czerniak and J. Pernak, ChemistrySelect, 2021, 6, 1994-2001.
40. J. Pernak, M. Niemezak, Ł. Chrzanowski, Ł. Ławniczak, P. Fochtman, K. Marcinkowska and T. Praczyk, Chemistry—A European Journal, 2016, 22, 12012-12021.
41. B. Grassiri, A. Mezzetta, G. Maisetta, C. Migone, A. Fabiano, S. Esin, L. Guazzelli, Y. Zambito, G. Batoni and A. M. Piras, Journal, 2023, 24.
42. H. K. Saeed, Y. Sutar, P. Patel, R. Bhat, S. Mallick, A. E. Hatada, D.-L. T. Koomoa, I. Lange and A. A. Date, ACS Omega, 2021, 6, 2626-2637.
43. D. J. Delgado-Diaz, B. Jesaveluk, J. A. Hayward, D. Tyssen, A. Alisoltani, M. Potgieter, L. Bell, E. Ross, A. Iranzadeh, I. Allah, S. Dabee, S. Barnabas, H. Gamieldien, J. M. Blackburn, N. Mulder, S. B. Smith, V. L. Edwards, A. D. Burgener, L.-G. Bekker, J. Ravel, J.-A. S. Passmore, L. Masson, A. C. Hearps and G. Tachedjian, Microbiome, 2022, 10, 141.
44. I. Schwecht, A. Nazli, B. Gill and C. Kaushic, Scientific Reports, 2023, 13, 20065.
45. R. Jackson, J. D. Maarsingh, M. M. Herbst-Kralovetz and K. Van Doorslaer, Curr Protoc Microbiol, 2020, 59, e129.
46. C. D. Shelton, E. Sing, J. Mo, N. G. Shealy, W. Yoo, J. Thomas, G. N. Fitz, P. R. Castro, T. T. Hickman, T. P. Torres, N. J. Foegeding, J. K. Zieba, M. W. Calcutt, S. G. Codreanu, S. D. Sherrod, J. A. McLean, S. H. Peck, F. Yang, N. O. Markham, M. Liu and M. X. Byndloss, Cell Host & Microbe, 2023, 31, 1604-1619.e1610.
47. M. F. Laursen, M. Sakanaka, N. von Burg, U. Mörbe, D. Andersen, J. M. Moll, C. T. Pekmez, A. Rivollier, K. F. Michaelsen, C. Mølgaard, M. V. Lind, L. O. Dragsted, T. Katayama, H. L. Frandsen, A. M. Vinggaard, M. I. Bahl, S. Brix, W. Agace, T. R. Licht and H. M. Roager, Nature Microbiology, 2021, 6, 1367-1382.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A composition comprising phenyllactic acid 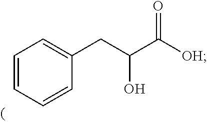 [SMILES: O=C(O)C(O)Cc1ccccc1]
PLA); including pharmaceutically acceptable salts, hydrates, solvates, stereoisomers, and/or deuterated analogues thereof.

2. The composition of claim 1, wherein the PLA is either the D enantiomer form of PLA, the L enantiomer form of PLA, or a mixture thereof.

3. The composition of claim 1, wherein the composition is configured for topical or mucosal delivery to a subject.

4. The composition of claim 3, wherein the subject is a human subject.

5. The composition of claim 1, wherein the composition is a liquid, wherein the PLA is solubilized within the liquid.

6. The composition of claim 1, wherein the composition is an ionic liquid, wherein the PLA is solubilized within the ionic liquid.

7. The composition of claim 1,
wherein the PLA is associated with choline, wherein the PLA is associated with choline 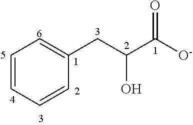 [SMILES: O=C([O-])C(O)Cc1ccccc1]
in the following manner: 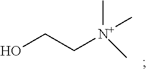
and/or
wherein the PLA is associated with carnitine, wherein the PLA is associated with 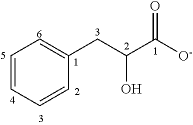 [SMILES: O=C([O-])C(O)Cc1ccccc1]
carnitine in the following manner:

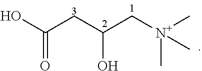

8. The composition of claim 7,
wherein the ratio of PLA to choline is approximately between 1:1 to 2:1;
wherein the ratio of PLA to carnitine is approximately between 1:1.

9. The composition of claim 7, wherein the composition is a liquid or ionic liquid, wherein the PLA associated with choline and/or carnitine is solubilized in the liquid or ionic liquid.

10. The composition of claim 1, wherein the composition is a pharmaceutical composition.

11. A method for increasing the amount of PLA within a subject's microbiome through administering to a subject a therapeutically effective amount of a composition recited in claim 1.

12. The method of claim 11, wherein the subject is a human subject (e.g., a female human subject).

13. The method of claim 11,
wherein the administering is a topical administering to the cervicovaginal mucosa of the subject, and optionally the administering is followed one or more of administering of a donor engraftment of the cervicovaginal mucosa, a vaginal microbiota transplant of the cervicovaginal mucosa, and/or aprobiotic colonization of the cervicovaginal mucosa.

14. The method of claim 11, wherein the subject is suffering from or at risk of suffering from a condition (e.g., sexually transmitted infections (e.g., syphilis, gonorrhea, chlamydia, trichomoniasis, hepatitis B, herpes simplex virus (HSV), HIV, and human papillomavirus (HPV)), urinary tract infections, and bacterial vaginosis) associated with inhibited or diminished PLA expression and/or activity and/or levels within the subject's microbiome.

15. The method of claim 14, wherein the microbiome is a cervico-vaginal microbiome.

16. The method of claim 11, wherein the subject is experiencing diminished or inhibited commensal vaginal Lactobacillus species activity and/or expression.

17. The method of claim 11, wherein the subject is experiencing diminished or inhibited commensal vaginal Lactobacillus species related PLA activity and/or expression.

18. The method of claim 11, wherein the subject is experiencing one or more of diminished or inhibited commensal vaginal Lactobacillus crispatus, and/or Lactobacillus paragasseri, and/or Lactobacillus iners activity and/or expression.

19. A method for elevating the richness and diversity of a subject's microbiome through administering to a subject a therapeutically effective amount of a composition recited in claim 1.

20. The method of claim 19, wherein the subject is a human subject (e.g., a female human subject).

21. The method of claim 19,
wherein the administering is a topical administering to the cervicovaginal mucosa of the subject, and optionally the administering is followed one or more of administering of a donor engraftment of the cervicovaginal mucosa, a vaginal microbiota transplant of the cervicovaginal mucosa, and/or aprobiotic colonization of the cervicovaginal mucosa.

22. The method of claim 19, wherein the subject is suffering from or at risk of suffering from a condition (e.g., sexually transmitted infections, urinary tract infections, and bacterial vaginosis) associated with inhibited or diminished PLA expression and/or activity and/or levels within the subject's microbiome.

23. The method of claim 22, wherein the microbiome is a cervico-vaginal microbiome.

24. The method of claim 19, wherein the subject is experiencing diminished or inhibited commensal vaginal Lactobacillus species activity and/or expression.

25. The method of claim 19, wherein the subject is experiencing diminished or inhibited commensal vaginal Lactobacillus species related PLA activity and/or expression.

26. The method of claim 19, wherein the subject is experiencing one or more of diminished or inhibited commensal vaginal Lactobacillus crispatus, and/or Lactobacillus paragasseri, and/or Lactobacillus iners activity and/or expression.

27. A method for treating or preventing microbiome dysbiosis in a subject through administering to a subject a therapeutically effective amount of a composition recited in claim 1.

28. The method of claim 27, wherein the subject is a human subject (e.g., a female human subject).

29. The method of claim 27,
wherein the administering is a topical administering to the cervicovaginal mucosa of the subject, and optionally the administering is followed one or more of administering of a donor engraftment of the cervicovaginal mucosa, a vaginal microbiota transplant of the cervicovaginal mucosa, and/or aprobiotic colonization of the cervicovaginal mucosa.

30. The method of claim 27, wherein the subject is suffering from or at risk of suffering from a condition (e.g., sexually transmitted infections (e.g., syphilis, gonorrhoea, chlamydia, trichomoniasis, hepatitis B, herpes simplex virus (HSV), HIV, and human papillomavirus (HPV)), urinary tract infections, and bacterial vaginosis) associated with inhibited or diminished PLA expression and/or activity and/or levels within the subject's microbiome.

31. The method of claim 30, wherein the microbiome is a cervico-vaginal microbiome.

32. The method of claim 27, wherein the subject is experiencing diminished or inhibited commensal vaginal Lactobacillus species activity and/or expression.

33. The method of claim 27, wherein the subject is experiencing diminished or inhibited commensal vaginal Lactobacillus species related PLA activity and/or expression.

34. The method of claim 27, wherein the subject is experiencing one or more of diminished or inhibited commensal vaginal Lactobacillus crispatus, and/or Lactobacillus paragasseri, and/or Lactobacillus iners activity and/or expression.

35. A method for treating or preventing a condition associated with diminished or inhibited commensal vaginal Lactobacillus species activity and/or expression in a subject through administering to a subject a therapeutically effective amount of a composition recited in claim 1.

36. The method of claim 35, wherein the subject is a human subject (e.g., a female human subject).

37. The method of claim 35,
wherein the administering is a topical administering to the cervicovaginal mucosa of the subject, and optionally the administering is followed one or more of administering of a donor engraftment of the cervicovaginal mucosa, a vaginal microbiota transplant of the cervicovaginal mucosa, and/or aprobiotic colonization of the cervicovaginal mucosa.

38. The method of claim 35, wherein the subject is suffering from or at risk of suffering from a condition (e.g., sexually transmitted infections (e.g., syphilis, gonorrhoea, chlamydia, trichomoniasis, hepatitis B, herpes simplex virus (HSV), HIV, and human papillomavirus (HPV)), urinary tract infections, and bacterial vaginosis) associated with inhibited or diminished PLA expression and/or activity and/or levels within the subject's microbiome.

39. The method of claim 38, wherein the microbiome is a cervico-vaginal microbiome.

40. The method of claim 35, wherein the subject is experiencing diminished or inhibited commensal vaginal Lactobacillus species related PLA activity and/or expression.

41. The method of claim 35, wherein the subject is experiencing one or more of diminished or inhibited commensal vaginal Lactobacillus crispatus, and/or Lactobacillus paragasseri, and/or Lactobacillus iners activity and/or expression.

42. A method for treating or preventing a sexually transmitted infection associated with diminished or inhibited commensal vaginal Lactobacillus species activity and/or expression in a subject through administering to a subject a therapeutically effective amount of a composition recited in claim 1.

43. The method of claim 42, wherein the subject is a human subject (e.g., a female human subject).

44. The method of claim 42,
wherein the administering is a topical administering to the cervicovaginal mucosa of the subject, and optionally the administering is followed one or more of administering of a donor engraftment of the cervicovaginal mucosa, a vaginal microbiota transplant of the cervicovaginal mucosa, and/or aprobiotic colonization of the cervicovaginal mucosa.

45. The method of claim 42, wherein the diminished or inhibited commensal vaginal Lactobacillus species activity and/or expression is in the subject's microbiome.

46. The method of claim 45, wherein the microbiome is a cervico-vaginal microbiome.

47. The method of claim 42, wherein the subject is experiencing diminished or inhibited commensal vaginal Lactobacillus species related PLA activity and/or expression.

48. The method of claim 42, wherein the subject is experiencing one or more of diminished or inhibited commensal vaginal Lactobacillus crispatus, and/or Lactobacillus paragasseri, and/or Lactobacillus iners activity and/or expression.

49. A method for treating or preventing a urinary tract infection associated with diminished or inhibited commensal vaginal Lactobacillus species activity and/or expression in a subject through administering to a subject a therapeutically effective amount of a composition recited in claim 1.

50. The method of claim 49, wherein the subject is a human subject (e.g., a female human subject).

51. The method of claim 49,
wherein the administering is a topical administering to the cervicovaginal mucosa of the subject, and optionally the administering is followed one or more of administering of a donor engraftment of the cervicovaginal mucosa, a vaginal microbiota transplant of the cervicovaginal mucosa, and/or aprobiotic colonization of the cervicovaginal mucosa.

52. The method of claim 49, wherein the diminished or inhibited commensal vaginal Lactobacillus species activity and/or expression is in the subject's microbiome.

53. The method of claim 52, wherein the microbiome is a cervico-vaginal microbiome.

54. The method of claim 49, wherein the subject is experiencing diminished or inhibited commensal vaginal Lactobacillus species related PLA activity and/or expression.

55. The method of claim 49, wherein the subject is experiencing one or more of diminished or inhibited commensal vaginal Lactobacillus crispatus, and/or Lactobacillus paragasseri, and/or Lactobacillus iners activity and/or expression.

56. A method for treating or preventing bacterial vaginosis associated with diminished or inhibited commensal vaginal Lactobacillus species activity and/or expression in a subject through administering to a subject a therapeutically effective amount of a composition recited in claim 1.

57. The method of claim 56, wherein the subject is a human subject (e.g., a female human subject).

58. The method of claim 57,
wherein the administering is a topical administering to the cervicovaginal mucosa of the subject, and optionally the administering is followed one or more of administering of a donor engraftment of the cervicovaginal mucosa, a vaginal microbiota transplant of the cervicovaginal mucosa, and/or aprobiotic colonization of the cervicovaginal mucosa.

59. The method of claim 58, wherein the diminished or inhibited commensal vaginal Lactobacillus species activity and/or expression is in the subject's microbiome.

60. The method of claim 56, wherein the microbiome is a cervico-vaginal microbiome.

61. The method of claim 56, wherein the subject is experiencing diminished or inhibited commensal vaginal Lactobacillus species related PLA activity and/or expression.

62. The method of claim 56, wherein the subject is experiencing one or more of diminished or inhibited commensal vaginal Lactobacillus crispatus, and/or Lactobacillus paragasseri, and/or Lactobacillus iners activity and/or expression.

63. A method for treating or preventing cervico-vaginal dysbiosis in a subject through administering to a subject a therapeutically effective amount of a composition recited in claim 1.

64. The method of claim 63, wherein the subject is a human subject (e.g., a female human subject).

65. The method of claim 63,
wherein the administering is a topical administering to the cervicovaginal mucosa of the subject, and optionally the administering is followed one or more of administering of a donor engraftment of the cervicovaginal mucosa, a vaginal microbiota transplant of the cervicovaginal mucosa, and/or aprobiotic colonization of the cervicovaginal mucosa.

66. The method of claim 63, wherein the subject is experiencing diminished or inhibited commensal vaginal Lactobacillus species activity and/or expression in the subject's microbiome.

67. The method of claim 66, wherein the microbiome is a cervico-vaginal microbiome.

68. The method of claim 63, wherein the subject is experiencing diminished or inhibited commensal vaginal Lactobacillus species related PLA activity and/or expression.

69. The method of claim 63, wherein the subject is experiencing one or more of diminished or inhibited commensal vaginal Lactobacillus crispatus, and/or Lactobacillus paragasseri, and/or Lactobacillus iners activity and/or expression.

70. The method of claim 63, wherein the cervico-vaginal dysbiosis is related to one or more of Gardnerella vaginalis, Prevotella bivia, and Atopobium vaginae.

71. A method for treating, preventing, and/or reducing dysbiotic vaginal bacteria in a subject through administering to a subject a therapeutically effective amount of a composition recited in claim 1,
wherein the administering is to the cervicovaginal mucosa of the subject,
wherein the administering is followed by donor engraftment of the cervicovaginal mucosa, vaginal microbiota transplant of the cervicovaginal mucosa, and/or probiotic colonization of the cervicovaginal mucosa.

72. The method of claim 71, wherein the subject is a human subject.

73. The method of claim 71, wherein the subject is a female human subject.

74. The method of claim 71, wherein the subject is experiencing diminished or inhibited commensal vaginal Lactobacillus species activity and/or expression in the subject's microbiome.

75. The method of claim 74, wherein the microbiome is a cervico-vaginal microbiome.

76. The method of claim 71, wherein the subject is experiencing diminished or inhibited commensal vaginal Lactobacillus species related PLA activity and/or expression.

77. The method of claim 71, wherein the subject is experiencing one or more of diminished or inhibited commensal vaginal Lactobacillus crispatus, and/or Lactobacillus paragasseri, and/or Lactobacillus iners activity and/or expression.

78. The method of claim 71, wherein the cervico-vaginal dysbiosis is related to one or more of Gardnerella vaginalis, Prevotella bivia, and Atopobium vaginae.

79. A method for treating, preventing, and/or reducing dysbiotic vaginal bacteria in a subject through administering to a subject one or more of a donor engraftment of the cervicovaginal mucosa, vaginal microbiota transplant of the cervicovaginal mucosa, and a probiotic colonization of the cervicovaginal mucosa,
wherein the donor engraftment, vaginal microbiota transplant, and the probiotic colonization each comprise a therapeutically effective amount of a composition recited in claim 1,
wherein the administering is to the cervicovaginal mucosa of the subject.

80. The method of claim 79, wherein the subject is a human subject.

81. The method of claim 79, wherein the subject is a female human subject.

82. The method of claim 79, wherein the subject is experiencing diminished or inhibited commensal vaginal Lactobacillus species activity and/or expression in the subject's microbiome.

83. The method of claim 82, wherein the microbiome is a cervico-vaginal microbiome.

84. The method of claim 79, wherein the subject is experiencing diminished or inhibited commensal vaginal Lactobacillus species related PLA activity and/or expression.

85. The method of claim 79, wherein the subject is experiencing one or more of diminished or inhibited commensal vaginal Lactobacillus crispatus, and/or Lactobacillus paragasseri, and/or Lactobacillus iners activity and/or expression.

86. The method of claim 79, wherein the cervico-vaginal dysbiosis is related to one or more of Gardnerella vaginalis, Prevotella bivia, and Atopobium vaginae.
